(19) United States
(12) Patent Application Publication
(10) Pub. No.: US 2020/0170894 A1
(43) Pub. Date: Jun. 4, 2020

(54) THICK CLEANSING COMPOSITIONS

(71) Applicant: L'OREAL, Paris (FR)

(72) Inventors: Angela PARK, Carlstadt, NJ (US); Jun LIANG, Staten Island, NY (US); Heather LEE, Wayne, NJ (US); Anthony POTIN, Nutley, NJ (US)

(73) Assignee: L'OREAL, Paris (FR)

(21) Appl. No.: 16/204,591

(22) Filed: Nov. 29, 2018

(51) Int. Cl. A61K 8/06; A61Q 5/02; A61Q 19/10; A61K 8/46; A61K 8/34; A61K 8/37; A61K 8/73; A61K 8/81; A61K 8/44; A61K 8/20; A61K 8/92; A61K 8/891; A61K 8/26; A61K 8/25; A61K 8/42; A61Q 5/12

(52) U.S. Cl. CPC A61K 8/062; A61Q 5/12; A61Q 19/10; A61K 8/466; A61K 8/463; A61K 8/342; A61K 8/375; A61K 8/737; A61K 8/8152; A61K 8/731; A61K 8/73; A61K 8/44; A61K 8/20; A61K 8/922; A61K 8/891; A61K 8/26; A61K 8/25; A61K 8/42; A61K 8/732; A61Q 5/02

(57) ABSTRACT

The present disclosure relates to a cleansing composition having a high viscosity and a cream-like texture. The composition includes anionic surfactants, fatty alcohols; glyceryl esters, cationic polymers, non-cationic polysaccharides, and optionally, one or more of conditioning agents, amphoteric surfactants, and opacifying agents. The composition can have a unique matte (non-pearlescent) appearance when an amphoteric surfactant such as a betaine is included. The thick consistency of the composition also allows for the suspension of particles from clays, powders, colorants and scrubs.

### FIELD OF THE INVENTION

The present disclosure relates to rinse-off cleansing compositions that include anionic surfactants, fatty alcohols, thickening agents, and emulsifiers. The cleansing compositions are particularly useful for cleaning and conditioning the body/skin and hair.

### BACKGROUND OF THE INVENTION

Most “dirt” contains traces of oil and grease, which stick to the surface of keratinous substrates such as the skin and hair. Rinsing with only water is not sufficient to adequately remove the oil and grease. The main functional ingredients in cleansing compositions are surfactants. Surfactants interact with water, thereby allowing it to “wet” surfaces more efficiently. The surfactant-water combination is then able to surround the specks of dirt and carry them away with rinsing. Agitation of the water solution, for example by rubbing hands together during washing or lathering a cleansing composition onto the keratinous substrate, also aids the process of removing dirt.

Conventional cleansing compositions such as shampoos and body/skin cleansers, for example, contain surfactants in various amounts. Anionic surfactants are typically included because they provide foaming to a composition. In some cleansing applications, higher viscosity is desired for the product's handling or ease of application. In addition, higher viscosity personal care products are more aesthetically appealing to many consumers.

The development of cleansing compositions has been driven by a need for certain performance properties and textures that consumers find desirable. For example, consumers seek cleansing compositions that foam and cleanse well, have a certain “thickness” (viscosity), and are mild to the skin and hair. The cleansing compositions should also rinse away from the body with ease. Thus, in addition to selecting the appropriate type of anionic surfactants, other ingredients may be needed in order to provide a desirable viscosity and texture to cleansing and personal care products. However, often, the addition of a particular component to a cleansing composition will enhance one desired property to the detriment of another desired property. It is therefore difficult to achieve a perfect balance of desirable performance properties.

### BRIEF SUMMARY OF THE INVENTION

The present disclosure relates to rinse-off cleansing compositions, that include anionic surfactants, in particular, non-sulfate anionic surfactants, thickening agent(s) such as cationic polymers and non-cationic polysaccharides, fatty alcohols, and emulsifiers such as glyceryl esters. Thee compositions have a high viscosity and a cream-like texture. They also have a unique matte (non-pearlescent) appearance.

Conventional hair cleansing compositions are formulated to be more fluid and/or either transparent or pearlescent in appearance. Thus, obtaining a rinse-off cleansing composition that is cream-like and matte in appearance is not as simple as merely increasing the total amount of anionic surfactants and/or adding thickening agents, fatty alcohols, and emulsifiers to a rinse-off cleansing composition. Typically, cleansing compositions may also be thickened by adding salts such as sodium chloride to the composition. The inventors discovered a unique balance of different surfactants that can be combined with thickening agents, fatty alcohols, and emulsifiers to form surprisingly effective cleansing compositions that are robust, stable, and safe, and have pleasant rheological properties and cream-like texture. The presence of salt such as sodium chloride (whether added to the composition or included in a raw material employed in the composition) was not necessary in order to impart the desirable viscosity or thickness to the composition.

The cleansing compositions of the instant case do not “weigh down” the hair. Rather, the compositions were found to impart good foaming, lather, distribution, detangling, suppleness, and smoothness to hair. Furthermore, the cleansing compositions of the instant case provide hair with desirable properties of improved manageability of hair, no buildup of product, and ease of styling and/or shaping.

The rinse-off cleansing compositions of the instant disclosure typically include:
(a) one or more anionic surfactants, salts thereof, and a mixture thereof;
(b) about 0.5 to about 3 wt. % of one or more fatty alcohols;
(c) about 0.1 to about 5 wt. % of one or more glyceryl esters;
(d) about 0.15 to about 1.2 wt. % of one or more cationic polymers selected from cationic cellulose derivatives, quaternized hydroxyethyl cellulose, cationic starch derivatives, cationic guar gum derivatives copolymers of acrylamide and dimethyldiallyammonium chloride, polyquaterniums, and a mixture thereof;
(e) about 0.5 to about 10 wt. % of one or more non-cationic polysaccharides selected from starches, gums, cellulose-based polymers, and a mixture thereof; and
(f) water;
wherein all weight percentages are based on the total weight of the cleansing composition.

The one or more anionic surfactants, salts thereof, and a mixture thereof are preferably selected from non-sulfate anionic surfactants.

In an embodiment, the one or more anionic surfactants, salts thereof, and a mixture thereof of the cleansing compositions of the present disclosure include non-sulfate anionic surfactants selected from acyl isethionate(s), acyl sarcosinate(s), salts thereof, and a mixture thereof.

In some embodiments, the compositions of the present disclosure further comprise one or more of conditioning agents, amphoteric surfactants, opacifying agents, nonionic surfactants, and mixtures thereof.

The present disclosure is also directed to methods of treating keratinous substrates such as hair and skin, the methods comprising the steps of:
(1) applying onto the keratinous substrates, the cleansing compositions of the present disclosure; and
(2) rinsing the cleansing composition from the keratinous substrates.

### DESCRIPTION OF THE INVENTION

The cleansing compositions of the instant disclosure typically include:
(a) one or more anionic surfactants, salts thereof, and a mixture thereof;
(b) about 0.5 to about 3 wt. % of one or more fatty alcohols;
(c) about 0.1 to about 5 wt. % of one or more glyceryl esters;
(d) about 0.15 to about 1.2 wt. % of one or more cationic polymers selected from cationic cellulose derivatives, quaternized hydroxyethyl cellulose, cationic starch derivatives, cationic guar gum derivatives copolymers of acrylamide and dimethyldiallyammonium chloride, polyquaterniums, and a mixture thereof;
(e) about 0.5 to about 10 wt. % of one or more non-cationic polysaccharides selected from starches, gums, cellulose-based polymers, and a mixture thereof; and
(f) water;
wherein all weight percentages are based on the total weight of the cleansing composition.

The cleansing compositions of the present disclosure can optionally contain one or more of conditioning agents, amphoteric surfactants, opacifying agents, nonionic surfactants, and mixtures thereof.

Anionic surfactants carry a negative charge on the polar head group. These surfactants are typically used for their detergency properties. They are highly effective at removing dirt and oil from the hair and scalp. Amphoteric (zwitterionic) surfactants are dual-charged (have both a positive and negative charge on the molecule). Many amphoteric surfactants display pH-dependent charge behavior, having one charge at a lower pH and the opposite charge at a higher pH. These types of surfactants tend to be mild both to skin and hair. They can also provide foam-boosting properties in combination with anionic surfactants, which enhances lather. Nonionic surfactants are those that have no (or very little) residual electric charge. These surfactants can perform a variety of functions, such as emulsion stabilization, mild detergency and viscosity modification.

The one or more anionic surfactants, salts thereof, and a mixture thereof are preferably selected from non-sulfate anionic surfactants.

In an embodiment, the one or more anionic surfactants, salts thereof, and a mixture thereof of the cleansing compositions of the present disclosure include non-sulfate anionic surfactants selected from acyl isethionate(s), acyl sarcosinate(s), salts thereof, and a mixture thereof.

In an embodiment, the cleansing compositions of the present disclosure further comprise one or more non-sulfate anionic surfactants selected from acyl amino acids other than acyl sarcosinates (for example, acyl taurates, acyl glycinates, acyl glutamates), alkyl sulfonates, alkyl sulfosuccinates, alkyl sulfoacetates, alkoxylated monoacids, salts thereof, and a mixture thereof

Non-limiting examples of alkyl sulfonates include C₈-C₁₆ alkyl benzene sulfonates, C₁₀-C₂₀ paraffin sulfonates, C₁₀-C₂₄ olefin sulfonates, and salts thereof. A non-limiting example of alkyl sulfonate salt is sodium C14-16 olefin sulfonate.

Non-limiting examples of alkyl sulfosuccinate salts include disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, and sodium dialkyl sulfosuccinate. A non-limiting example of an alkyl sulfoacetate salt includes sodium lauryl sulfoacetate.

In some embodiments, the one or more anionic surfactants are preferably selected from acyl isethionates, acyl sarcosinates, salts thereof, and a mixture thereof.

In some instances, a plurality of (two or more, or three or more) non-sulfate anionic surfactants is preferred, for example, a plurality of non-sulfate anionic surfactants selected from acyl isethionates, acyl sarcosinates, salts thereof, and a mixture thereof. In other instances, additional non-sulfate anionic surfactants may be included such as alkyl sulfonates, alkyl sulfosuccinates, alkyl sulfoacetates, salts thereof, and a mixture thereof. Similarly, in some cases, a plurality of (two or more, or three or more) acyl amino acid surfactants may be included, for example, a plurality of acyl amino acid surfactants selected from acyl taurates, acyl sarcosinates, acyl glycinates, acyl glutamates, and a mixture thereof.

In some embodiments of the instant disclosure, the one or more anionic surfactants are present in the compositions of the present disclosure in amounts of from about 4 to about 20 wt. %, preferably about 6 to about 15 wt. %, more preferably about 8 to about 13 wt. %, based on the total weight of the composition.

Non-limiting examples of fatty alcohols include cetearyl alcohol, decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, cholesterol, cis4-t-butylcyclohexanol, and a mixture thereof. In some cases, the fatty alcohols are those selected from the group consisting of cetearyl alcohol, cetyl alcohol, stearyl alcohol, isostearyl alcohol, oleyl alcohol, isotridecyl alcohol, and a mixture thereof.

In some embodiments of the instant disclosure, the one or more fatty alcohols are present in the compositions of the present disclosure in amounts of from about 0.5 to about 3 wt. %, preferably about 1 to about 2.5 wt. %, more preferably about 1.5 to about 2 wt. %, based on the total weight of the composition.

The one or more glyceryl esters may include glyceryl stearate, glyceryl isostearate, glyceryl oleate, glyceryl caprate, and glyceryl caprylate.

In an embodiment, the one or more glyceryl esters are selected from glyceryl stearate, glyceryl isostearate, and a mixture thereof.

In some embodiments of the instant disclosure, the one or more glyceryl esters are present in the compositions of the present disclosure in amounts of from about 0.1 to about 5 wt. %, preferably about 0.25 to about 4.5 wt. %, more preferably about 0.5 to about 4 wt. %, even more preferably about 0.75 to about 3.5 wt. %, based on the total weight of the composition.

In some embodiments, the one or more cationic polymers are selected from cationic guar gum derivatives.

In an embodiment, the one or more cationic polymers are selected from hydroxypropyl guar hydroxypropyltrimonium chloride, guar hydroxypropyltrimonium chloride, and a mixture thereof.

In some embodiments of the instant disclosure, the one or more cationic polymers are present in the compositions of the present disclosure in amounts of from about 0.15 to about 1.2 wt. %, preferably about 0.2 to about 1 wt. %, more preferably about 0.25 to about 0.8 wt. %, even more preferably about 0.3 to about 0.7 wt. %, based on the total weight of the composition.

The one or more non-cationic polysaccharides include nonionic polysaccharides and anionic polysaccharides.

In an embodiment, the one or more non-cationic polysaccharides are selected from starches, gums, and a mixture thereof.

In some embodiments of the instant disclosure, the one or more non-cationic polysaccharides are present in the compositions of the present disclosure in amounts of from about 0.5 to about 10 wt. %, preferably about 0.75 to about 8 wt. %, more preferably about 1 to about 6 wt. %, even more preferably about 1 to about 4 wt. %, based on the total weight of the composition.

In some embodiments, the compositions of the present disclosure further comprise one or more conditioning agents (g) selected from cationic polymers other than the cationic polymers described above, non-silicone fatty compounds, silicones, starch hydrolysates, cationic proteins, cationic protein hydrolysates, oils, ester oils, alkyl amines, and a mixture thereof.

Non-limiting examples of conditioning agents include non-silicone fatty compounds, silicones, starch hydrolysates, oils, ester oils, alkyl amines, and a mixture thereof.

In an embodiment, the one or more conditioning agents are selected from starch hydrolysates, preferably, hydrogenated starch hydrolysates, present in an amount of from about 1 to about 15 wt. %, preferably about 3 to about 14 wt. %, and more preferably about 5 to about 12 wt. %, based on the total weight of the composition.

In an embodiment, the one or more conditioning agents include non-silicone fatty compounds selected from plant/vegetable oils and butters, mineral oils, ester oils, and a mixture thereof and present in an amount of from about 0.05 to about 5 wt. %, preferably about 0.1 to about 3 wt. %, and more preferably about 0.25 to about 2 wt. %, based on the total weight of the composition.

In an embodiment, the one or more conditioning agents include silicones present in an amount of from about 0.05 to about 5 wt. %, preferably about 0.1 to about 3 wt. %, and more preferably about 0.5 to about 2 wt. %, based on the total weight of the composition.

In some embodiments, the compositions of the present disclosure further comprise one or more amphoteric surfactants (h).

Amphoteric surfactants include, for example, betaines, alkyl sultaines, alkyl amphoacetates, amphoproprionates, salts thereof, and a mixture thereof. Non-limiting examples betaines include coco betaine, cocamidopropyl betaine, lauryl betaine, laurylihydroxy sulfobetaine, lauryldimethyl betaine, behenyl betaine, capryl/capramidopropyl betaine, stearyl betaine, and a mixture thereof. Non-limiting examples of sultaines include cocamidopropyl hydroxysultaine, lauryl hydroxysultaine, and a mixture thereof. A non-limiting example of an alkyl amphoacetate salt includes sodium lauroamphoacetate. Non-limiting examples of amphopropionates include cocoamphopropionate, cornamphopropionate, salts thereof, and a mixture thereof.

In an embodiment, the one or more amphoteric surfactants include betaines selected from coco-betaine, cocamidopropyl betaine, and a mixture thereof,

In an embodiment, the one or more amphoteric surfactants are present in an amount of from about 0.5 to about 10 wt. %, preferably about 1 to about 8 wt. %, and more preferably about 2 to about 6 wt. %, based on the total weight of the composition.

In some embodiments, the compositions of the present disclosure further comprise one or more opacifying agent (i).

In an embodiment, the one or more opacifying agent (i) is selected from clay, talc, bentonite and a mixture thereof.

In an embodiment, the one or more opacifying agent (i) is present in an amount of from about 0.1 to about 8 wt. %, or about 0.25 to about 6 wt. %, or about 0.5 to about 4 wt. %, or about 0.1 to about 3 wt. %, based on the total weight of the composition.

The cleansing compositions may optionally include nonionic surfactants. Nonionic surfactants include alkyl polyglucosides, alkoxylated fatty alcohols, fatty amindes, polyethoxylated fatty amides, polyglycerolated fatty amides, alkoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides, and polyglycerolated C₈-C₄₀ alcohols.

Non-limiting examples of alkyl polyglucosides include lauryl glucoside, octyl glucoside, decyl glucoside, coco glucoside, and a mixture thereof.

Non-limiting examples of alkoxylated fatty alcohols include laureth-2, oleth-2, ceteareth-2, laneth-2, laureth-3, oleth-3, ceteareth-3, laureth-4, oleth-4, ceteareth-4, laneth-4, laureth-5, oleth-5, ceteareth-5, laneth-5, deceth-4, deceth-7, laureth-7, oleth-7, coceth-7, ceteth-7, ceteareth-7, C11-15 pareth-7, laureth-9, oleth-9, ceteareth-9, laureth-10, oleth-10, beheneth-10, ceteareth-10, laureth-12, ceteareth-12, trideceth-12, ceteth-15, laneth-15, ceteareth-15, laneth-16, ceteth-16, oleth-16, steareth-16, oleth-20, ceteth-20, ceteareth-20, laneth-20, steareth-21, ceteareth-23, ceteareth-25, ceteareth-27, and a mixture thereof.

The cleansing compositions may optionally include other thickening agents such as carboxylic acid polymers (e.g., carbomer), crosslinked polyacrylate polymers, polyacrylamide polymers, polysaccharides, gums, and a mixture thereof.

In an embodiment, the cleansing compositions of the instant disclosure are emulsions, in particular, oil in water emulsions.

The cleansing compositions of the instant disclosure are particularly useful for cleansing and conditioning keratinous substrates such as hair and skin. The compositions exhibit good cleansing ability, lather, foaming and foam stability, and conditioning properties. Additionally, when used on hair, the cleansing compositions provide a variety of desirable benefits to the hair, for example, smoothness, detangling, suppleness, manageability, and ease of styling and/or shaping. Accordingly, the cleansing compositions may be used in methods for cleansing hair, methods of conditioning hair, and methods for imparting smoothness, detangling, suppleness, and/or manageability to hair, as well as ease of styling and/or shaping of hair.

### Anionic Surfactants

The anionic surfactants may be chosen from non-sulfate anionic surfactants.

### Non-Sulfate Anionic Surfactants

In some instances, the non-sulfate anionic surfactant(s) are the predominant type of surfactant in the surfactant system (i.e., there is a higher percentage of non-sulfate anionic surfactant(s) than any other single surfactant type in the cleansing composition). Moreover, in some instances, the total amount of non-sulfate anionic surfactants in the surfactant system is higher than the total amount of all other surfactant types in the surfactant system including amphoteric surfactants, nonionic surfactants, and any sulfate-based anionic surfactant that may be present. In other words, the phrase “all other surfactants” means any and all surfactants in the cleansing composition other than anionic surfactants.

The total amount of non-sulfate anionic surfactants in the cleansing compositions can vary but typically ranges from about 4 to about 20 wt. %, based on the total weight of the cleansing composition. In some cases, the total amount of non-sulfate anionic surfactants in the cleansing composition may be from about 4 to about 20 wt. %, from about 5 to about 20 wt. %, from about 5 to about 18 wt. %, from about 5 to about 15 wt. %, from about 6 to about 15 wt. %, from about 7 to about 15 wt. %, from about 7 to about 14 wt. %, or from about 7 to about 13 wt. %, based on the total weight of the cleansing composition. In some embodiments, the total amount of non-sulfate anionic surfactants in the cleansing compositions can vary from about 6 to about 15 wt. %, from about 7 to about 14 wt. %, or from about 8 to about 13 wt. %, based on the total weight of the cleansing composition, including all ranges and subranges therebetween.

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. Non-limiting examples of useful non-sulfate anionic surfactants are provided below.

Acyl Isethionates

Non-limiting examples of useful acyl isethionates and their salts include those of formula (I) and (II):

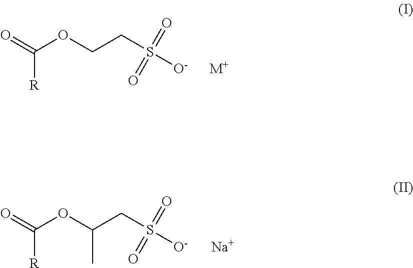

wherein R, R¹, R² and R³ are each independently selected from H or an alkyl chain having 1-24 carbon atoms, said chain being saturated or unsaturated, linear or branched, and X is COO⁻ or SO₃⁻. Sodium is shown as the cation in formula (I) but the cation for both formula (I) and formula (II) may be an alkali metal ion such as sodium or potassium, ammonium ions, or alkanolammonium ions such as monoethanolammonium or triethanolammonium ions. Non-limiting examples of acyl isethionates include sodium cocoyl isethionate, sodium lauroyl isethionate, sodium lauroyl methyl isethionate, and sodium cocoyl methyl isethionate.

The total amount of acyl isethionate(s) in the cleansing composition, if present, may vary but is typically from about 4 to about 20 wt. %, based on the total weight of the cleansing composition. In some instance, the total amount of acyl isethionate(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

In various embodiments, the total amount of acyl isethionate(s) in the cleansing composition, if present, is typically at about 6 wt. %, 6.5 wt. %, 7 wt. %, 7.5 wt. %, 8 wt. %, 8.5 wt. %, 8.8 wt. %, 9 wt. %, 9.5 wt. %, 10 wt. %, 10.5 wt. %, 11 wt. %, 11.4 wt. %, 12 wt. %, 12.5 wt. %, 13 wt. %, 13.5 wt. %, 14 wt. %, 14.5 wt. %, or 15 wt. %, based on the total weight of the cleansing composition.

Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

The total amount of acyl sarcosinate(s) in the cleansing composition, if present, may vary but is typically from about 4 to about 20 wt. %, based on the total weight of the cleansing composition. In some instance, the total amount of acyl sarcosinate(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition.

In some embodiments, the total amount of acyl sarcosinates in the cleansing composition, if present, is from about 0.5 to about 10 wt. %, based on the total weight of the cleansing composition. In some instance, the total amount of acyl sarcosinates in the cleansing composition is from about 0.5 to about 10 wt. %, about 1 to about 8 wt. %, about 1.5 to about 7 wt. %, about 1.75 to about 6 wt. %, 2 to about 5 wt. %, about 2 to about 4 wt. %, about 2.5 to about 4 wt. %, about 2.5 to about 3.5 wt. %, or about 2.7 to about 3 wt. %, based on the total weight of the cleansing composition, including all ranges and subranges therebetween.

In various embodiments, the total amount of acyl sarcosinate(s) in the cleansing composition, if present, is typically at about 0.5 wt. %, 0.75 wt. %, 1 wt. %, 1.25 wt. %, 1.5 wt. %, 1.75 wt. %, 2 wt. %, 2.25 wt. %, 2.5 wt. %, 2.7 wt. %, 3 wt. %, 3.25 wt. %, 3.5 wt. %, 3.75 wt. %, 4 wt. %, 4.25 wt. %, 4.5 wt. %, 4.75 wt, % or 5 wt. %, based on the total weight of the cleansing composition.

Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (III) is particularly useful.

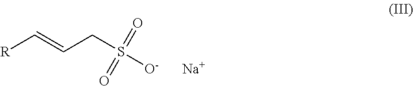

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (III) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates are particularly preferred. A non-limiting but particularly useful example of a C₁₀-C₂₄ olefin sulfonate that can be used in the instant compositions is sodium C14-16 olefin sulfonate.

The total amount of alkyl sulfonate(s) in the cleansing composition, if present, may vary but is typically from about 4 to about 20 wt. %, based on the total weight of the cleansing composition. In some instance, the total amount of alkyl sulfonate(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

Alkyl Sulfosuccinates

Non-limiting examples of useful sulfosuccinates and their salts include those of formula (IV):

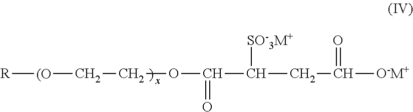

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

The total amount of alkyl sulfosuccinate(s) in the cleansing composition, if present, may vary but is typically from about 4 to about 20 wt. %, based on the total weight of the cleansing composition. In some instance, the total amount of alkyl sulfosuccinate(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

Alkyl Sulfoacetates

Non-limiting examples of alkyl sulfoacetates and their salts include, for example, alkyl sulfoacetates such as C4-C18 fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

The total amount of alkyl suoflacetate(s) in the cleansing composition, if present, may vary but is typically from about 4 to about 20 wt. %, based on the total weight of the cleansing composition. In some instance, the total amount of alkyl sulfoacetate(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

Alkoxylated Monoacids

Non-limiting examples of alkoxylated monoacids include compounds corresponding to formula (V):

RO[CH2O]u[(CH2)xCH(R′)(CH2)y(CH2)zO]v[CH2CH2O]wCH2COOH (V)

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z>0;

Compounds corresponding to formula (V) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (V), R is linear or branched, acyclic or cyclic, saturated or unsaturated, aliphatic or aromatic, substituted or unsubstituted. Typically, R is a linear or branched, acyclic C6-40 alkyl or alkenyl group or a C1-40 alkyl phenyl group, more typically a C8-22 alkyl or alkenyl group or a C4-18 alkyl phenyl group, and even more typically a C12-18 alkyl group or alkenyl group or a C6-16 alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

The total amount of alkoxylated monoacids in the cleansing composition, if present, may vary but is typically from about 4 to about 20 wt. %, based on the total weight of the cleansing composition. In some instance, the total amount of alkoxylated monoacids in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (VI):

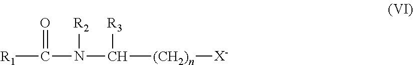

wherein R, R¹, R² and R³ are each independently selected from H or an alkyl chain having 1-24 carbon atoms, said chain being saturated or unsaturated, linear or branched, and X is COO⁻ or SO₃⁻.

The total amount of acyl amino acid(s) in the cleansing composition, if present, may vary but is typically from about 4 to about 20 wt. %, based on the total weight of the cleansing composition. In some instance, the total amount of acyl amino acid(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition.

Acyl Taurates

Non-limiting examples of acyl taurates include those of formula (VII):

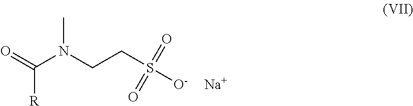

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate and sodium methyl cocoyl taurate.

The total amount of acyl taurate(s) in the cleansing composition, if present, may vary but is typically from about 4 to about 20 wt. %, based on the total weight of the cleansing composition. In some instance, the total amount of acyl taurate(s) in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

Acyl Glycinates

Non-limiting examples of useful acyl glycinates include those of formula (VIII):

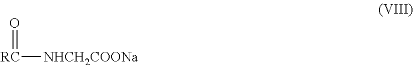

wherein R is an alkyl chain of 8 to 16 carbon atoms. Sodium is shown as the cation in the above formula (VIII) but the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

The total amount of acyl glycinates in the cleansing composition, if present, may vary but is typically from about 4 to about 20 wt. %, based on the total weight of the cleansing composition. In some instance, the total amount of acyl glycinates in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

Acyl Glutamates

Non-limiting examples of useful acyl glutamates include those of formula (IX):

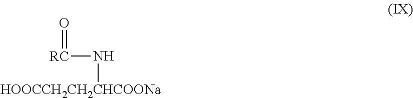

wherein R is an alkyl chain of 8 to 16 carbon atoms. Sodium is shown as the cation in the above formula (IX) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

The total amount of acyl glutamates in the cleansing composition, if present, may vary but is typically from about 4 to about 20 wt. %, based on the total weight of the cleansing composition. In some instance, the total amount of acyl glutamates in the cleansing composition is from about 4 to about 20 wt. %, about 5 to about 20 wt. %, about 5 to about 18 wt. %, about 5 to about 15 wt. %, about 6 to about 15 wt. %, about 6 to about 14 wt. %, about 6 to about 13 wt. %, about 7 to about 12 wt. %, or about 7 to about 11.4 wt. %, based on the total weight of the cleansing composition, including all ranges and subranges therebetween.

In an embodiment, the non-sulfate anionic surfactants of the compositions of the present disclosure is selected from one or more acyl isethionates, their salts, and a mixture thereof.

In an embodiment, the one or more acyl isethionates, their salts, and a mixture thereof are selected from sodium cocoyl isethionate, sodium lauroyl methyl isethionate, sodium cocoyl methyl isethionate, sodium lauroyl isethionate, and a mixture thereof.

In an embodiment, the non-sulfate anionic surfactants of the compositions of the present disclosure is selected from one or more acyl sarcosinates, their salts, and a mixture thereof.

In an embodiment, the one or more acyl sarcosinates, their salts, and a mixture thereof are selected from sodium cocoyl sarcosinate, sodium lauroyl sarcosinate, potassium lauroyl sarcosinate, potassium cocoyl sarcosinate, sodium myristoyl sarcosinate, sodium oleoyl sarcosinate, sodium palmitoyl sarcosinate, ammonium lauroyl sarcosinate, and a mixture thereof.

In some cases, a plurality of non-sulfate anionic surfactants is preferred. For example, two or more, or three or more, non-sulfate anionic surfactants may be selected from acyl isethionates, acyl sarcosinates, alkyl sulfonates, alkyl sulfosuccinates, alkyl sulfoacetates, alkoxylated monoacids, acyl amino acids such as acyl taurates, acyl glycinates, acyl glutamates, salts thereof, and a mixture thereof.

One embodiment of the compositions of the present disclosure includes a combination of non-sulfate anionic surfactants chosen from one or more acyl isethionatse (and/or salts thereof) and one or more acyl sarcosinatse (and/or salts thereof).

One embodiment of the compositions of the present disclosure includes a combination of non-sulfate anionic surfactants chosen from one or more acyl isethionates (and/or salts thereof), one or more acyl sarcosinates (and/or salts thereof), and one or more non-sulfate anionic surfactants and/or salts thereof selected from alkyl sulfonates, alkyl sulfosuccinates, alkyl sulfoacetates, alkoxylated monoacids, acyl amino acids such as acyl taurates, acyl glycinates, acyl glutamates, and mixtures thereof.

### Fatty Alcohols

The compositions of the present disclosure comprise one or more fatty alcohols. The fatty alcohols that may be used in the composition of the disclosure include, but are not limited to, non-alkoxylated, saturated or unsaturated, linear or branched, and have from 6 to 60 carbon atoms, such as from 8 to 30 carbon atoms.

The fatty alcohols of the present disclosure are chosen from solid and liquid fatty alcohols.

The saturated liquid fatty alcohols can be branched. They can optionally comprise, in their structure, at least one aromatic or non-aromatic ring. They can be acyclic.

The unsaturated liquid fatty alcohols exhibit, in their structure, at least one double or triple bond and preferably one or more double bonds. When several double bonds are present, there are preferably 2 or 3 of them and they can be conjugated or unconjugated. These unsaturated fatty alcohols can be linear or branched. They can optionally comprise, in their structure, at least one aromatic or non-aromatic ring. They can be acyclic. Among the liquid unsaturated fatty alcohols, oleyl alcohol, linoleyl alcohol, linolenyl alcohol and undecylenyl alcohol may be mentioned.

Liquid fatty alcohols may be selected, for example, from octyldodecanol, 2-butyloctanol, 2-hexyldecanol, 2-undecylpentadecanol, oleyl alcohol or linoleyl alcohol, isostearyl alcohol, and mixtures thereof.

Solid fatty alcohols may be crystalline, amorphous or pasty. The solid fatty alcohols of the present invention are solid at room temperature (25 degrees centigrade) and at atmospheric pressure (1 atm) and are insoluble in water (i.e. they have a solubility in water of less than 1% by weight and preferably less than 0.5% by weight, at 25° C. and 1 atm) and are soluble, under the same temperature and pressure conditions, in at least one organic solvent (for example ethanol, chloroform, benzene or liquid petroleum jelly) to at least 1% by weight.

In an embodiment, the solid fatty alcohols preferably have a melting point of greater than or equal to 28° C. and have a viscosity, at a temperature of 40° C. and at a shear rate of 1 s⁻¹, of greater than or equal to 1 Pa·s.

In an embodiment, the melting point of the fatty alcohols ranges from 30° C. to 250° C., such as from 32° C. to 150° C. or such as from 35° C. to 150° C.

The melting points may be measured by DSC or on a Kofler bench. The melting point may be measured by differential calorimetric analysis (DSC) with a temperature rise of 10° C. per minute. The melting point is then the temperature corresponding to the top of the melting endotherm peak obtained during the measurement.

The viscosity measurements may be taken at a temperature of about 40° C. using an RS600 rheometer from Thermoelectron.

The solid fatty alcohols of the present invention are chosen from saturated or unsaturated, linear or branched, preferably linear and saturated, (mono) alcohols comprising from 6 to 60 carbon atoms, such as from 10 to 50 carbon atoms, or such as from 12 to 24 carbon atoms.

The solid fatty alcohols preferably have the structure of formula: R—OH in which R especially denotes a C6-C60, for example, C8-C60, preferably C10-050 or even C12-C30 alkyl group, R possibly being substituted with one or more hydroxyl groups, R possibly being branched. The solid fatty alcohols of the invention may be non-oxyalkylenated and/or non-glycerolated. These fatty alcohols may be constituents of animal or plant waxes.

The solid fatty alcohol may represent a mixture of fatty alcohols, which means that several species of fatty alcohol may coexist, in the form of a mixture, in a commercial product. One example of such a commercial product is cetearyl alcohol, a mixture of cetyl alcohol and stearyl alcohol, commercially available under the trade name of LANETTE O OR from the company BASF. Cetyl alcohol may also be commercially available under the tradename of LANETTE 16 from the company BASF.

In an embodiment, the solid fatty alcohols of the present invention may be chosen from myristyl alcohol, cetyl alcohol, stearyl alcohol, cetearyl alcohol, and mixtures thereof, octyldodecanol, 2-butyloctanol, 2-hexyldecanol, 2-undecylpentadecanol, oleic alcohol, linoleic alcohol, behenyl alcohol, and mixtures thereof.

Other suitable examples of the solid fatty alcohol of the present invention include branched solid fatty alcohols chosen from 2-dodecylhexadecanol, 2-tetradecyl-1-octadecanol, 2-tetradecyl-1-eicosanol, 2-hexadecyl-1-octadecanol and 2-hexadecyl-1-eicosanol, and mixtures thereof.

In an embodiment of the present invention, the fatty alcohol is chosen from non-alkoxylated, saturated or unsaturated, linear or branched fatty alcohol having from 6 to 60 carbon atoms is chosen from cetyl alcohol, stearyl alcohol, cetearyl alcohol (mixture of cetyl alcohol and stearyl alcohol), octyldodecanol, 2-butyloctanol, 2-hexyldecanol, 2-undecylpentadecanol, oleic alcohol, linoleic alcohol, behenyl alcohol, 2-dodecylhexadecanol, 2-tetradecyl-1-octadecanol, 2-tetradecyl-1-eicosanol, 2-hexadecyl-1-octadecanol and 2-hexadecyl-1-eicosanol, octyldodecanol, 2-butyloctanol, 2-hexyldecanol, 2-undecylpentadecanol, oleyl alcohol or linoleyl alcohol, isostearyl alcohol, and mixtures thereof

In an embodiment of the present invention, the fatty alcohol is chosen from cetyl alcohol.

In an embodiment of the present invention, the fatty alcohol is chosen from cetearyl alcohol.

In an embodiment of the present invention, the fatty alcohol comprises cetyl alcohol and cetearyl alcohol.

In an embodiment of the present invention, the fatty alcohol comprises cetyl alcohol and stearyl alcohol.

In an embodiment of the present invention, the fatty alcohol is chosen from cetyl alcohol, stearyl alcohol, cetearyl alcohol, and mixtures thereof.

In an embodiment, the fatty alcohols of the present invention are chosen from liquid fatty alcohol, solid fatty alcohols, and mixtures thereof.

The fatty alcohol(s) may be present in the cleansing composition according to the invention in an amount of from about 0.5 to about 3 wt. % by weight, such as from about 0.75 to about 2.75 wt. % by weight, from about % to about 2.5 wt. % by weight, from about 1.25 to about 2.5 wt. % by weight, from about 1.5 to about 2.25 wt. %, from about 1.5 to about 2 wt. %, or from about 1.7 to about 2 wt. %, based on the total weight of the cleansing composition, including all ranges and subranges therebetween.

In various embodiments, the total amount of the fatty alcohol(s), if present, is typically at about 0.5 wt. %, 0.75 wt. %, 1 wt. %, 1.25 wt. %, 1.5 wt. %, 1.7 wt. %, 1.75 wt. %, 2 wt. %, 2.25 wt. %, 2.5 wt. %, 2.75 wt. %, or 3 wt. %, based on the total weight of the cleansing composition.

### Glyceryl Esters

The one or more glyceryl esters of the compositions of the present invention may be selected include glyceryl stearate, glyceryl isostearate, glyceryl oleate, glyceryl caprate, glyceryl caprylate, and a mixture thereof.

In an embodiment, the one or more emulsifiers are selected from glyceryl stearate, glyceryl isostearate, and a mixture thereof.

The emulsifiers may be present in the cleansing composition according to the invention in an amount of from about 0.1 to about 5 wt. %, about 0.25 to about 4.5 wt. %, about 0.5 to about 4 wt. %, about 0.75 to about 3.5 wt. %, or about 1 to about 3 wt. %, based on the total weight of the cleansing composition, including all ranges and subranges therebetween.

In various embodiments, the total amount of the glyceryl esters, if present, is typically at about 0.1 wt. %, 0.25 wt. %, 0.5 wt. %, 0.75 wt. %, 1 wt. %, 1.25 wt. %, 1.5 wt. %, 1.7 wt. %, 1.75 wt. %, 2 wt. %, 2.25 wt. %, 2.5 wt. %, 2.75 wt. %, 3 wt. %, 3.25 wt. %, 3.5 wt. %, 3.75 wt. %, 4 wt. %, 4.25 wt. %, 4.5 wt. %, 4.75 wt. %, or 5 wt. %, based on the total weight of the cleansing composition.

### Thickening Agents—Cationic Polymers and Non-Cationic Polysaccharides

The cleansing compositions contain thickening agents (also referred to as thickeners or viscosity modifying agents). Thickening agents can be water-soluble, and increase the viscosity of water when dispersed/dissolved in water. The compositions may be heated and cooled, or neutralized, if necessary. Thickening agents may be dispersed/dissolved in an aqueous solvent that is soluble in water, e.g., ethyl alcohol when it is dispersed/dissolved in water.

### Cationic Polymers

Suitable examples of cationic polymers are those from selected from cationic cellulose derivatives, quaternized hydroxyethyl cellulose, cationic starch derivatives, cationic guar gum derivatives (hydroxypropyl guar hydroxypropyltrimonium chloride, guar hydroxypropyltrimonium chloride), copolymers of acrylamide and dimethyldiallyammonium chloride, polyquaterniums, and a mixture thereof.

In an embodiment, the cationic polymer is selected from cationic guar gum derivatives or guar gums containing cationic trialkylammonium groups, their salts, and a mixture thereof.

Suitable examples of cationic guar gum derivatives are hydroxypropyl guar hydroxypropyltrimonium chloride, guar hydroxypropyltrimonium chloride, guar gums modified with a 2,3-epoxypropyltrimethylammonium salt (for example, chloride). and a mixture thereof.

Such products are sold especially under the trade names JAGUAR C-13-S guar hydroxypropyltrimonium chloride), JAGUAR C135, JAGUAR C15, JAGUAR C17 and JAGUAR C162 (hydroxypropyl guar hydroxypropyltrimonium chloride) by the company Rhodia (Solvay).

Guar hydroxypropyltrimonium chloride may also be sold under the tradename of N-HANCE CG13 by the company Ashland.

In an embodiment, the cationic polymer is selected from cationic cellulose derivatives, quaternized hydroxyethyl cellulose, and a mixture thereof.

In an embodiment, the cationic polymer is selected from polyqauternium compounds.

In an embodiment, the cationic polymer is selected from hydroxypropyl guar hydroxypropyltrimonium chloride, guar hydroxypropyltrimonium chloride, and a mixture thereof.

The total amount of the cationic polymer (s) in the cleansing composition, if present, is from about 0.15 to about 1.2 wt. %, based on the total weight of the cleansing composition. In some instance, the total amount of the cationic polymer (s) in the cleansing composition is from about 0.15 to about 1.2 wt. %, about 0.2 to about 1.2 wt. %, about 0.25 to about 1 wt. %, about 0.25 to about 0.9 wt. %, 0.25 to about 0.8 wt. %, about 0.3 to about 0.8 wt. %, about 0.3 to about 0.7 wt. %, about 0.35 to about 0.65 wt. %, or about 0.4 to about 0.6 wt. %, based on the total weight of the cleansing composition, including all ranges and subranges therebetween.

In various embodiments, the total amount of the cationic polymer (s) in the cleansing composition, if present, is typically at about 0.15 wt. %, 0.2 wt. %, 0.25 wt. %, 0.3 wt. %, 0.35 wt. %, 0.4 wt. %, 0.45 wt. %, 0.5 wt. %, 0.55 wt. %, 0.6 wt. %, 0.65 wt. %, 0.7 wt. %, 0.75 wt. %, 0.8 wt. %, 0.85 wt. %, 0.9 wt. %, 0.95 wt. %, 1 wt. %, 1.1 wt,% or 1.2 wt. %, based on the total weight of the cleansing composition.

### Non-Cationic Polysaccharides

The non-cationic polysaccharide(s) may be selected from polysaccharides that are nonionic or anionic. Such polysaccharides may be selected from starches, gums and cellulose-based polymers, and a mixture thereof.

A wide variety of polysaccharides can be useful as the non-cationic polysaccharide(s) herein. “Polysaccharides” refer to gelling agents that contain a backbone of repeating sugar (i.e., carbohydrate) units.

Suitable examples of starches include modified starches, starch-based polymers, methylhydroxypropyl starch, potato starch, wheat starch, rice starch, starch crosslinked with octenyl succinic anhydride (sold under the name Dry-Flo by National Starch), starch oxide, dialdehyde starch, dextrin, British gum, acetyl starch, starch phosphate, carboxymethyl starch, hydroxyethyl starch, and hydroxypropyl starch.

Suitable examples of cellulose-based polymers include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename NATROSOL CS Plus from Aqualon Corporation.

Other useful polysaccharides include scleroglucans comprising a linear chain of (1-3) linked glucose units with a (1-6) linked glucose every three units, a commercially available example of which is CLEAROGEL. CS11 from Michel Mercier Products Inc.

Non-limiting examples of gums include acacia, agar, algin, alginic acid, ammonium alginate, amylopectin, calcium alginate, calcium carrageenan, carnitine, carrageenan, dextrin, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, biosacharide gum, and mixtures thereof.

In an embodiment, the non-cationic polysaccharide(s) is selected from hydroxypropyl starch phosphate, potato starch (modified or unmodified), wheat starch, rice starch, hydroxyethyl cellulose, guar gum, hydroxypropyl guar, xanthan gum, sclerotium gum, and a mixture thereof.

In an embodiment, the non-cationic polysaccharide(s) is selected from hydroxypropyl starch phosphate. Hydroxypropyl starch phosphate is sold under the tradename of STRUCTURE ZEA by the company Akzo Nobel.

In an embodiment, the non-cationic polysaccharide(s) is selected from hydroxyethyl cellulose.

In an embodiment, the non-cationic polysaccharide(s) is selected from xanthan gum.

The total amount of the non-cationic polysaccharide(s) in the cleansing composition, if present, is from about 0.5 to about 10 wt. %, based on the total weight of the cleansing composition. In some instance, the total amount of the non-cationic polysaccharide(s) in the cleansing composition is from about 0.5 to about 10 wt. %, about 0.75 to about 9 wt. %, about 0.75 to about 8 wt. %, about 0.75 to about 7 wt. %, 0.75 to about 6 wt. %, about 1 to about 5 wt. %, about 1 to about 4 wt. %, or about 1 to about 3 wt. %, based on the total weight of the cleansing composition, including all ranges and subranges therebetween.

In various embodiments, the total amount of the non-cationic polysaccharide(s) in the cleansing composition, if present, is typically at about 0.5 wt. %, 0.75 wt. %, 0.8 wt. %, 0.85 wt. %, 0.9 wt. %, 0.95 wt. %, 1 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, or 4 wt. %, based on the total weight of the cleansing composition.

### Water

The compositions according to various embodiments of the disclosure are aqueous. Water can be present in total amounts of about 90% or less, such as from about 90% to about 40% by weight, or about 90%, 80%, 70%, 60%, 50%, or 40% by weight, based on the total weight of the composition, including all ranges and subranges therebetween. Additionally, water can be present in the compositions of the present disclosure in the amount of from about 50 to about 90 wt. % by weight, from about 55% to about 85% by weight, or from about 60% to about 80% by weight, based on the total weight of the compositions.

In other embodiments, water can be present in the compositions of the present disclosure in the amount of at least about 95%, 90%, 80%, 75%, 70%, 65%, 60%, 55%, 50%, 45%, or 40%, by weight, based on the total weight of the compositions.

### Water-Soluble Solvents

The cleansing compositions may optionally include one or more water-soluble solvents. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water soluble solvents has a solubility of at least 60%, 70%, 80%, or 90%.

The total amount of water-soluble solvents in the cleansing compositions, if present, may vary but are typically in an amount of about 0.01 to about 25 wt. %, based on the total weight of the cleansing composition. In some instances, the total amount of water-soluble solvents may be from about 0.01 to about 20 wt. %, about 0.01 to about 15 wt. %, about 0.01 to about 10 wt. %, about 0.01 to about 5 wt. %, about 0.1 to about 25 wt. %, about 0.1 to about 20 wt. %, about 0.1 to about 15 wt. %, about 0.1 to about 10 wt. %, about 0.1 to about 5 wt. %, based on the total weight of the cleansing composition, including all ranges and subranges therebetween.

Non-limiting examples of water-soluble solvents include, for example, organic solvents selected from glycerin, alcohols (for example, C₁₋₁₂, C₁₋₁₀, C₁₋₈, or C₁₋₄ alcohols), polyols (polyhydric alcohols), glycols, and a mixture thereof.

As examples of organic solvents, non-limiting mention can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

### Amphoteric Surfactants

The cleansing compositions of the present disclosure may further comprise one or more amphoteric surfactants. The total amount of amphoteric surfactant(s) in the cleansing compositions may vary but is typically from about 1 to about 25 wt. %, based on the total weight of the cleansing composition. In some instance, the total amount of amphoteric surfactant(s) in the cleansing composition is from about 2 to about 20 wt. %, from about 2 to about 15 wt. %, from about 2 to about 10 wt. %, from about 2 to about 8 wt. %, from about 2 to about 5 wt. %, from about 2 to about 4 wt. %, or from about 2.5 to about 3.5 wt. %, based on the total weight of the cleansing composition, including all ranges and subranges therebetween.

Useful amphoteric surfactants include betaines, alkyl sultaines, alkyl amphoacetates, alkyl amphoproprionates, and mixtures thereof. Non-limiting examples of useful amphoteric surfactants are provided below.

(1) Betaines

Useful betaines include those of the following formulae (XIIIa-XIIId):

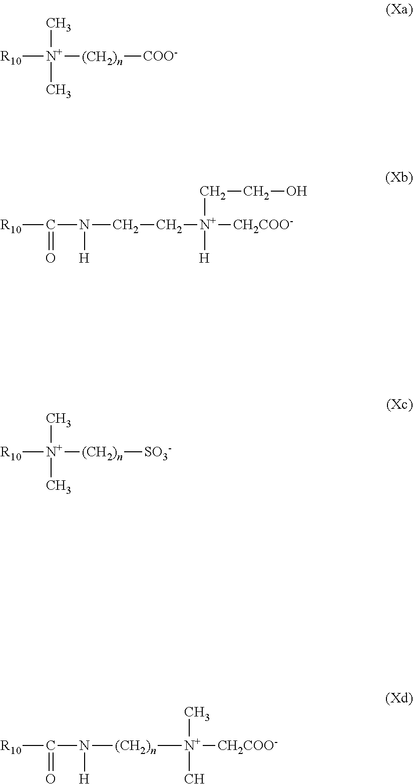

wherein R₁₀ is an alkyl group having 8-18 carbon atoms; and n is an integer from 1 to 3.

Particularly useful betaines include, for example, coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, cocamidopropyl betaine, behenyl betaine, capryl/capramidopropyl betaine, and lauryl betaine, and mixtures thereof. Particularly preferred betaines include coco betaine and cocamidopropyl betaine.

The total amount of betaines in the cleansing composition, if present, may vary but is typically from about 1 to about 10 wt. %, based on the total weight of the cleansing composition. In some instance, the total amount of betaines(s) in the cleansing composition is from about 1 to about 10 wt. %, from about 1.5 to about 8 wt. %, from about 1.5 to about 7 wt. %, from about 1.5 to about 6 wt. %, from about 2 to about 5 wt. %, from about 2.5 to about 4 wt. %, or from about 2.5 to about 3.5 wt. %, based on the total weight of the cleansing composition, including all ranges and subranges therebetween.

(2) Alkyl Sultaines

Non-limiting examples of alkyl sultaines include hydroxyl sultaines of formula (XIV)

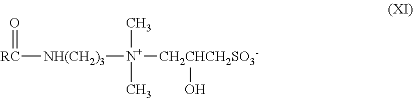

wherein R is an alkyl group having 8-18 carbon atoms. More specific examples include, but are not limited to cocamidopropyl hydroxysultaine, lauryl hydroxysultaine, and a mixture thereof.

The total amount of alkyl sultaines in the cleansing composition, if present, may vary but is typically from about 0.01 to about 25 wt. %, based on the total weight of the cleansing composition. In some instance, the total amount of alkyl sultaines(s) in the cleansing composition is from about 0.01 to about 20 wt. %, about 0.01 to about 15 wt. %, about 0.01 to about 10 wt. %, about 0.01 to about 5 wt. %, about 0.1 to about about 25 wt. %, about 0.1 to about 20 wt. %, about 0.1 to about 15 wt. %, or about 0.1 to about 10 wt. %, or about 0.1 to about 5 wt. %, based on the total weight of the cleansing composition, including all ranges and subranges therebetween.

(3) Alkyl Amphoacetates and Alkyl Amphodiacetates

Useful alkyl amphoacetates and alkyl amphodiacetates include those of Formula (XV) and (XVI):

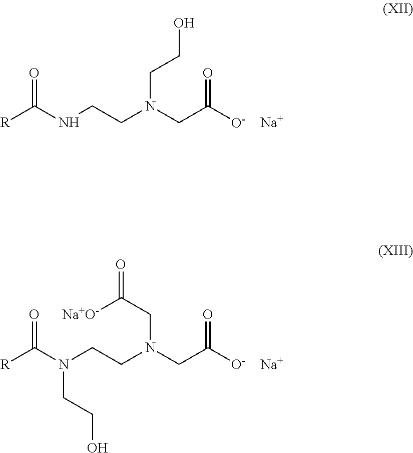

wherein R is an alkyl group having 8-18 carbon atoms. Sodium is shown as the cation in the above formulae above but the cation may be an alkali metal ion such as sodium or potassium, ammonium ions, or alkanolammonium ions such as monoethanolammonium or triethanolammonium ions. A more specific, but non-limiting example, is sodium lauroamphoacetate.

The total amount of alkyl amphoacetates and/or alkyl amphodiacetates in the cleansing composition, if present, may vary but is typically from about 0.01 to about 25 wt. %, based on the total weight of the cleansing composition. In some instance, the total amount of alkyl amphoacetates and/or alkyl amphodiacetates in the cleansing composition is from about 0.01 to about 20 wt. %, from about 0.01 to about 15 wt. %, from about 0.01 to about 10 wt. %, from about 0.01 to about 5 wt. %, from about 0.1 to about 25 wt. %, from about 0.1 to about 20 wt. %, from about 0.1 to about 15 wt. %, or from about 0.1 to about 10 wt. %, or about 0.1 to about 5 wt. %, based on the total weight of the cleansing composition, including all ranges and subranges therebetween.

(4) Alkyl Amphopropionates

Non-limiting examples of alkyl amphopropionates include cocoamphopropionate, cornamphopropionatecaprylamphopropionate, cornamphopropionate, caproamphopropionate, oleoamphopropionate, isostearoamphopropionate, stearoamphopropionate, lauroamphopropionate, salts thereof, and a mixture thereof.

The total amount of alkyl amphopropionates in the cleansing composition, if present, may vary but is typically from about 0.01 to about 25 wt. %, based on the total weight of the cleansing composition. In some instance, the total amount of amphopropionates in the cleansing composition is from about 0.01 to about 20 wt. %, about 0.01 to about 15 wt. %, about 0.01 to about 10 wt. %, about 0.01 to about 5 wt. %, about 0.1 to about 25 wt. %, about 0.1 to about 20 wt. %, about 0.1 to about 15 wt. %, or about 0.1 to about 10 wt. %, or about 0.1 to about 5 wt. %, based on the total weight of the cleansing composition, including all ranges and subranges therebetween.

### Conditioning Agents

The cleansing compositions of the present disclosure may further comprise one or more conditioning agents. The total amount of conditioning agent(s) in the cleansing compositions can vary but is typically from about 0.1 to about 10 wt. %, based on the total weight of the cleansing composition. In some instances, the total amount of conditioning agent(s) in the cleansing compositions is from about 0.05 to about 20 wt. %, from about 0.05 to about 15 wt. %, from about 0.1 to about 14 wt. %, from about 0.15 to about 13 wt. %, from about 0.2 to about 12 wt. %, from about 0.25 to about 12 wt. %, from about 0.3 to about 12 wt. %, from about 0.4 to about 12 wt. %, or from about 0.5 to about 12 wt. %, based on the total weight of the cleansing composition, including all ranges and subranges therebetween.

Non-limiting examples of conditioning agents include cationic polymers other than the cationic polymers described above, non-silicone fatty compounds, silicones, starch hydrolysates, cationic proteins, cationic protein hydrolysates, oils, ester oils, alkyl amines, and a mixture thereof.

(1) Cationic Conditioning Polymers

The cationic conditioning polymers may be homopolymers or formed from two or more types of monomers. The molecular weight of the polymer may be between 5,000 and 10,000,000, typically at least 10,000, and preferably in the range 100,000 to about 2,000,000. These polymers will typically have cationic nitrogen containing groups such as quaternary ammonium or protonated amino groups, or a mixture thereof.

The cationic charge density is suitably at least 0.1 meq/g, preferably above 0.8 or higher. In some instances, the cationic charge density does not exceed 3 meq/g, or does not exceed 2 meq/g. The charge density can be measured using the Kjeldahl method and can be within the above limits at the desired pH of use, which will in general be from about 3 to 9 and preferably between 4 and 8.

The cationic nitrogen-containing group will generally be present as a substituent on a fraction of the total monomer units of the cationic conditioning polymer. Thus when the polymer is not a homopolymer it can contain spacer non-cationic monomer units.

Suitable cationic conditioning polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C₁-C₇ alkyl groups, more preferably C₁-C₃ alkyl groups. Other suitable spacers include vinyl esters, vinyl alcohol, maleic anhydride, propylene glycol and ethylene glycol.

The cationic amines can be primary, secondary or tertiary amines, depending upon the particular species and the pH of the composition.

Amine substituted vinyl monomers and amines can be polymerized in the amine form and then converted to ammonium by quaternization.

Suitable cationic amino and quaternary ammonium monomers include, for example, vinyl compounds substituted with dialkyl amincalkyl acrylate, dialkylamino alkylmethacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidine, e.g., alkyl vinyl imidazolium, and quaternized pyrrolidine, e.g., alkyl vinyl imidazolium, alkyl vinyl pyridinium, alkyl vinyl pyrrolidine salts. The alkyl portions of these monomers are preferably lower alkyls such as the C₁-C₃ alkyls, more preferably C₁ and C₂ alkyls.

Suitable amine-substituted vinyl monomers include dialkylaminoalkyl acrylate, dialkylaminoalkyl methacrylate, dialkylaminoalkyl acrylamide, and dialkylaminoalkyl methacrylamide, wherein the alkyl groups are preferably C₁-C₇ hydrocarbyls, more preferably C₁-C₃, alkyls.

The cationic conditioning polymers can comprise mixtures of monomer units derived from amine- and/or quaternary ammonium-substituted monomer and/or compatible spacer monomers.

(2) Non-Silicone Fatty Compounds

The term “non-silicone fatty compound” means a fatty compound that does not containing any silicon atoms (Si). Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. Non-limiting examples of the fatty alcohols, fatty acids, fatty alcohol derivatives, and fatty acid derivatives are found in International Cosmetic Ingredient Dictionary, Sixteenth Edition, 2016, which is incorporated by reference herein in its entirety.

Fatty alcohols useful herein include those having from about 10 to about 30 carbon atoms, from about 12 to about 22 carbon atoms, and from about 16 to about 22 carbon atoms. These fatty alcohols can be straight or branched chain alcohols and can be saturated or unsaturated. Nonlimiting examples of fatty alcohols include decyl alcohol, undecyl alcohol, dodecyl, myristyl, cetyl alcohol, stearyl alcohol, isostearyl alcohol, isocetyl alcohol, behenyl alcohol, linalool, oleyl alcohol, cholesterol, cis4-t-butylcyclohexanol, myricyl alcohol and a mixture thereof. In some cases, the fatty alcohols are those selected from the group consisting of cetyl alcohol, stearyl alcohol, isostearyl alcohol, oleyl alcohol, and a mixture thereof.

Fatty acids useful herein include those having from about 10 to about 30 carbon atoms, from about 12 to about 22 carbon atoms, and from about 16 to about 22 carbon atoms. These fatty acids can be straight or branched chain acids and can be saturated or unsaturated. Also included are diacids, triacids, and other multiple acids which meet the carbon number requirement herein. Also included herein are salts of these fatty acids. Nonlimiting examples of fatty acids include lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Non-limiting examples of polyglycerol esters of fatty acids include those of the following formula:

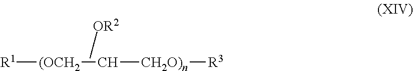

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. For example, nonionic polyglycerol esters of fatty acids include polyglyceryl-5 laurate,

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

In some cases, the one or more fatty compounds may be one or more high melting point fatty compounds. A high melting point fatty compound is a fatty compound having a melting point of 25° C. Even higher melting point fatty compounds may also be used, for example, fatty compounds having a melting point of 40° C. or higher, 45° C. or higher, 50° C. or higher. The high melting point fatty compound may be selected from fatty acids, fatty alcohol derivatives, fatty acid derivatives, and mixtures thereof. Nonlimiting examples of the high melting point compounds are found in the International Cosmetic Ingredient Dictionary, Sixteenth Edition, 2016, which is incorporated by reference herein in its entirety.

In some instances, the non-silicone fatty compounds include one or more waxes. The waxes generally have a melting point of from 35-120° C., at atmospheric pressure. Non-limiting examples of waxes in this category include for example, synthetic wax, ceresin, paraffin, ozokerite, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, sunflower seed wax (Helianthus annuus), acacia decurrents flower wax, or a mixture thereof.

In one embodiment, the cleaning composition includes waxes. Mention may be made, among the waxes capable of being used as non-silicone fatty compounds, of animal waxes, such as beeswax; vegetable waxes, such as sunflower seed (Helianthus annuus), carnauba, candelilla, ouricury or japan wax or cork fibre or sugarcane waxes; mineral waxes, for example paraffin or lignite wax or microcrystalline waxes or ozokerites; synthetic waxes, including polyethylene waxes, and waxes obtained by the Fischer-Tropsch synthesis.

In some instance, the non-silicone fatty compounds include one or more non-silicone oils. The term “oil” as used herein describes any material which is substantially insoluble in water. Suitable non-silicone oils include, but are not limited to, natural oils, such as coconut oil; hydrocarbons, such as mineral oil and hydrogenated polyisobutene; fatty alcohols, such as octyldodecanol; esters, such as C12-C15 alkyl benzoate; diesters, such as propylene dipelarganate; and triesters, such as glyceryl trioctanoate. Suitable low viscosity oils have a viscosity of 5-100 mPas at 25° C., and are generally esters having the structure RCO—OR′ wherein RCO represents the carboxylic acid radical and wherein OR′ is an alcohol residue. Examples of these low viscosity oils include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, or combinations thereof. The high viscosity oils generally have a viscosity of 200-1,000,000, or 100,000-250,000, mPas at 25° C. Such oils include castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Mineral oils, such as liquid paraffin or liquid petroleum, or animal oils, such as perhydrosqualene or arara oil, or alternatively of vegetable oils, such as sweet almond, calophyllum, palm, castor, avocado, jojoba, olive or cereal germ oil, may be utilized. It is also possible to use esters of these oils, e.g., jojoba esters. Also useful are esters of lanolic acid, of oleic acid, of lauric acid, of stearic acid or of myristic acid; esters of alcohols, such as oleyl alcohol, linoleyl or linolenyl alcohol, isostearyl alcohol or octyldodecanol; and/or acetylglycerides, octanoates, decanoates or ricinoleates of alcohols or of polyalcohols. It is alternatively possible to use hydrogenated oils which are solid at 25° C., such as hydrogenated castor, palm or coconut oils, or hydrogenated tallow; mono-, di-, tri- or sucroglycerides; lanolins; or fatty esters which are solid at 25° C.

(3) Silicones

The conditioning agent(s) of the cleansing compositions may optionally include one or more silicones. Nonetheless, as mentioned throughout the instant disclosure, in some instances the cleansing compositions are free or essentially free of silicones. In other words, one or more of the following silicones may be optionally included or optionally excluded from the cleansing compositions.

Silicones include, but are not limited to, polyorganosiloxanes, polyalkylsiloxanes, polyarylsiloxanes, polyalkarylsiloxanes, polyestersiloxanes, and a mixture thereof. Non-limiting examples include dimethicone, cyclomethicone (cyclopentasiloxane), amodimethicone, trimethyl silyl amodimethicone, phenyl trimethicone, trimethyl siloxy silicate, polymethylsilsesquioxane and a mixture thereof.

In some instances, the cleansing compositions include (or exclude) one or more silicones selected from the group consisting of polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxane (amodimethicone), non-ionic dimethicone copolyols, dimethicone copolyol esters, dimethicone copolyol quaternium nitrogen containing compounds, dimethicone copolyol phosphate esters, and mixtures thereof.

The cleansing compositions may include (or exclude) one or more silicone oils, for example one or more non-phenyl silicone oils and/or one or more phenyl silicone oils. The silicone oil is preferably apolar. An “apolar silicone oil” is intended to denote a silicon oil that does not comprise any ionic or ionisable group(s), and preferably does not comprise any oxyalkylenated (C2-C4) unit(s) (preferably oxyethylene, oxypropylene), or glycerol unit(s).

Representative examples of non-volatile non-phenyl silicone oils which may be mentioned include polydimethylsiloxanes; alkyl dimethicones; vinylmethyl methicones; and also silicones modified with aliphatic groups and/or with functional groups such as hydroxyl, thiol and/or amine groups. It should be noted that “dimethicone” (INCI name) corresponds to a poly(dimethylsiloxane) (chemical name), which is particularly preferred in some instances.

The non-volatile non-phenyl silicone oil is preferably chosen from non-volatile dimethicone oils. In particular, these oils can be chosen from the following non-volatile oils:
polydimethylsiloxanes (PDMSs),
PDMSs comprising aliphatic groups, in particular alkyl or alkoxy groups, which are pendent and/or at the end of the silicone chain, these groups each comprising from 2 to 24 carbon atoms. By way of example, mention may be made of the cetyl dimethicone sold under the commercial reference ABIL WAX 9801 from Evonik Goldschmidt,
PDMSs comprising aliphatic groups, or functional groups such as hydroxyl, thiol and/or amine groups, [
polyalkylmethylsiloxanes substituted with functional groups such as hydroxyl, thiol and/or amine groups,
polysiloxanes modified with fatty acids, fatty alcohols or polyoxyalkylenes, and mixtures thereof.

Preferably, these non-volatile non-phenyl silicone oils are chosen from polydimethylsiloxanes; alkyl dimethicones and also PDMSs comprising aliphatic groups, in particular C₂-C₂₄ alkyl groups, and/or functional groups such as hydroxyl, thiol and/or amine groups.

The non-phenyl silicone oil may be chosen in particular from silicones of the following formula:

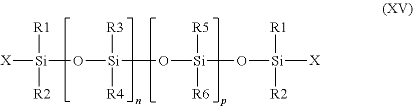

in which:

R₁, R₂, R₅ and R₆ are, together or separately, an alkyl radical containing 1 to 6 carbon atoms,

R₃ and R₄ are, together or separately, an alkyl radical containing from 1 to 6 carbon atoms, a vinyl radical, an amine radical or a hydroxyl radical,

X is an alkyl radical containing from 1 to 6 carbon atoms, a hydroxyl radical or an amine radical,

n and p are integers chosen so as to have a fluid compound, in particular of which the viscosity at 25° C. is between 9 centistokes (cSt) and 800 000 (cSt).

As non-volatile non-phenyl silicone oils which can be used according to the invention, mention may be made of those for which:
the substituents R₁ to R₆ and X represent a methyl group, and p and n are such that the viscosity is 500 000 cSt, for example the product sold under the name SE30 by the company General Electric, the product sold under the name AK 500000 by the company Wacker, the product sold under the name Mirasil DM 500 000 by the company Bluestar, and the product sold under the name Dow Corning 200 Fluid 500 000 cSt by the company Dow Corning,
the substituents R₁ to R₆ and X represent a methyl group, and p and n are such that the viscosity is 60 000 cSt, for example the product sold under the name Dow Corning 200 Fluid 60 000 CS by the company Dow Corning, and the product sold under the name Wacker Belsil DM 60 000 by the company Wacker,
the substituents R₁ to R₆ and X represent a methyl group, and p and n are such that the viscosity is 100 cSt or 350 cSt, for example the products sold respectively under the names Belsil DM100 and Dow Corning 200 Fluid 350 CS by the company Dow Corning,
the substituents R₁ to R₆ represent a methyl group, the group X represents a hydroxyl group, and n and p are such that the viscosity is 700 cSt, for example the product sold under the name Baysilone Fluid T0.7 by the company Momentive.

(4) Cationic Proteins and Cationic Protein Hydrolysates

Cationic proteins and cationic protein hydrolysates can be derived from animals, for example from collagen, milk, or keratin, from plants, for example from wheat, corn, rice, potatoes, soy, moringa, or almonds, from marine life forms, for example from fish collagen or algae, or from biotechnologically obtained protein hydrolysates. Those cationic protein hydrolysates may have a molecular weight from 100 to 25,000 dalton, from 250 to 5,000 dalton, or from 250 to 1000 dalton. Also to be understood as cationic protein hydrolysates are quaternized amino acids and mixtures thereof. Quaternization of the protein hydrolysates or of the amino acids is often carried out by means of quaternary ammonium salts such as, for example, N,N-dimethyl-N-(n-alkyl)-N-(2-hydroxy-3-chloro-n-propyl)ammonium halides. Typical examples that may be mentioned of cationic protein hydrolysates and derivatives according to the present invention are the products listed under the INCI names in the “International Cosmetic Ingredient Dictionary and Handbook,” (seventh edition 1997, The Cosmetic, Toiletry, and Fragrance Association), which is incorporated herein by reference in its entirety. Non-limiting examples of Cationic protein hydrolysates include: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-Hydrolyzed Collagen, Quaternium-79 Hydrolyzed Keratin, Quaternium-79 Hydrolyzed Milk Protein, Quaternium-79 Hydrolyzed Soy Protein, Quaternium-79 Hydrolyzed Wheat Protein.

Plant-based cationic proteins and cationic protein hydrolysates include but are not limited to those based on wheat, rice, corn, soy, almond, or moring, etc. Examples of cationic protein hydrolysates based on wheat include the commercial products GLUADIN WQ, GLUADIN WQT, and the HYDROTRITICUM series of the Croda company.

(5) Starch Hydrolysates

In an embodiment, the one or more conditioning agent(s) is selected from starch hydrolysates and preferably, from hydrogenated starch hydrolysates (hydrogenated corn syrup).

The total amount of the starch hydrolysates in the cleansing compositions, if present, is from about 0.5 to about 15 wt. %, from about 1 to about 14 wt. %, from about 1 to about 13 wt. %, from about 2 to about 12 wt. %, from about 2 to about 11 wt. %, from about 2.5 to about 11 wt. %, from about 3 to about 10.5 wt. %, from about 3.5 to about 10.5 wt. %, or from about 5 to about 10.5 wt. %, based on the total weight of the cleansing composition, including all ranges and subranges therebetween.

In some embodiments, the total amount of the starch hydrolysates in the cleansing compositions, if present, is at about 1 wt. %, 1.5 wt. %, 2 wt. %, 2.5 wt. %, 3 wt. %, 3.5 wt. %, 4 wt. %, 4.5 wt. %, 5 wt. %, 5.5 wt. %, 6 wt. %, 6.5 wt. %, 7 wt. %, 7.5 wt. %, 8 wt. %, 8.5 wt. %, 9 wt. %, 9.5 wt. %, or 10 wt. %, based on the total weight of the cleansing compositions.

(6) Miscellaneous Conditioning Agents

Many conditioning agents are known to those skilled in the art and need not be specifically listed herein. Nonetheless, a non-limiting example of miscellaneous conditioning agents include alkyl amines, such as mono-long alkyl amines, and ester oils. Mono-long alkyl amines include those having one long alkyl chain of preferably from 12 to 30 carbon atoms, more preferably from 16 to 24 carbon atoms, still more preferably from 18 to 22 alkyl group. Mono-long alkyl amines include mono-long alkyl amidoamines. Primary, secondary, and tertiary fatty amines are useful.

Non-limiting examples of alkyl amines include brassicamidopropyl dimethylamine, stearyl dimethyl amine, and stearamidopropyl dimethylamine.

Useful are tertiary amido amines having an alkyl group of from about 12 to about carbons. Exemplary tertiary amido amines include: brassicamidopropyl dimethylamine, stearamidopropyldimethylamine, stearamidopropyldiethylamine, stearamidoethyldiethylamine, stearamido-ethyldimethylamine, palmitamidopropyldimethylamine, palmitamidopropyldiethylamine, palmitamidoethyldiethylamine, palmitamidoethyldimethylamine, behenamidopropyl-dimethylamine, behenamidopropyldiethylamine, behenamidoethyldiethylamine, behenamidoethyldimethylamine, arachidamidopropyldimethylamine, arachidamido-propyldiethylamine, arachidamidoethyldiethylamine, arachidamidoethyldimethylamine, diethylaminoethylstearamide.

These amines may be used in combination with acids such as 1-glutamic acid, lactic acid, hydrochloric acid, malic acid, succinic acid, acetic acid, fumaric acid, tartaric acid, citric acid, 1-glutamic hydrochloride, maleic acid, and mixtures thereof; more preferably 1-glutamic acid, lactic acid, and citric acid.

As already noted, the conditioning agent may be an ester oil. Ester oils include, but are not limited to, fatty esters having at least 10 carbon atoms. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.)

The ester oil may for example be chosen from: monoesters comprising at least 18 carbon atoms and even more particularly containing between 18 and 40 carbon atoms in total, in particular monoesters of formula R₁COOR₂ in which R₁ represents a linear or branched, saturated or unsaturated or aromatic fatty acid residue comprising from 4 to 40 carbon atoms and R₂ represents a hydrocarbon-based chain that is in particular branched, containing from 4 to 40 carbon atoms, on condition that the sum of the carbon atoms of the radicals R₁ and R₂ is greater than or equal to 18, for instance Purcellin oil (cetostearyl octanoate), isononyl isononanoate, C₁₂ to C₁₅ alkyl benzoate, 2-ethylhexyl palmitate, octyldodecyl neopentanoate, 2-octyldodecyl stearate, 2-octyldodecyl erucate, isostearyl isostearate, C₁₂-C₁₅ alkyl benzoates such as 2-octyldodecyl benzoate, alcohol or polyalcohol octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate or 2-octyldodecyl myristate.

Preferably, they are esters of formula R₁COOR₂ in which R₁ represents a linear or branched fatty acid residue comprising from 4 to 40 carbon atoms and R₂ represents a hydrocarbon-based chain that is in particular branched, containing from 4 to 40 carbon atoms, R₁ and R₂ being such that the sum of the carbon atoms of the radicals R₁ and R₂ is greater than or equal to 18.

### Opacifying Agents

The cleansing compositions of the present disclosure may further comprise one or more opacifying agents selected from clay such as kaolin, talc, bentonite and a mixture thereof.

The total amount of opacifying agent(s) in the cleansing composition, if present, may vary but is typically from about 0.1 to about 8 wt. %, based on the total weight of the cleansing composition. In some instance, the total amount of opacifying agent(s) in the cleansing composition is from about 0.1 to about 8 wt. %, about 0.25 to about 7 wt. %, about 0.25 to about 6 wt. %, about 0.25 to about 5 wt. %, about 0.25 to about 4.5 wt. %, about 0.25 to about 4 wt. %, about 0.5 to about 4 wt. %, or about 0.5 to about 3.5 wt. %, or about 0.5 to about 3 wt. %, based on the total weight of the cleansing composition.

### Nonionic Surfactants

The cleansing compositions may optionally include one or more nonionic surfactants. The total amount of nonionic surfactant(s), if present, can vary but may be in an amount of from about 0.01 to about 25 wt. %, based on the total weight of the cleansing composition. In some instance, the total amount of nonionic surfactant(s) in the cleansing composition is from about 0.01 to about 20 wt. %, from about 0.01 to about 15 wt. %, about 0.01 to about 10 wt. %, from about 0.01 to about 5 wt. %, from about 0.1 to about 25 wt. %, from about 0.1 to about 20 wt. %, from about 0.1 to about 15 wt. %, or from about 0.1 to about 10 wt. %, or about 0.1 to about 5 wt. %, based on the total weight of the cleansing composition.

The nonionic surfactant(s) can be, for example, selected from alkyl polyglucosodes, fatty amide, alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides, and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

Useful alkyl polyglucosides include those having the following formula (XII):

R¹—O—(R²O)ₙ—Z(x) (XVI)

wherein R¹ is an alkyl group having 8-18 carbon atoms;

R² is an ethylene or propylene group;

Z is a saccharide group with 5 to 6 carbon atoms;

n is an integer from 0 to 10; and

x is an integer from 1 to 5.

Useful alkylpolyglucosides include lauryl glucoside, octyl glucoside, decyl glucoside, coco glucoside, caprylyl/capryl glucoside, sodium lauryl glucose carboxylate, and a mixture thereof. Typically, the alkyl polyglucoside compound is selected from the group consisting of lauryl glucoside, decyl glucoside, coco glucoside, and a mixture thereof. In some instances, decyl glucoside is particularly preferred.

### Film-Forming Polymers

The cleansing compositions of the instant disclosure do not require film-forming polymers (including anionic, amphoteric, and nonionic film-forming polymers). However, one or more filming-forming polymers may optionally be included. Therefore, the cleansing compositions may optionally include or exclude (may be free or essentially free of) one or more film forming polymers. Non-limiting examples of film-forming polymers that may optionally be included or excluded from the cleansing compositions include vinyl polymers, polyesters, polyamides, polyureas, and a mixture thereof. The one or more film-forming polymers may be polyethyleneimine, polylysine, polyvinyl alcohols, poly(hydroxyethyl (meth)acrylate), hydroxyalkylcelluloses, polyacrylic acid, polyvinylimidazoles, polypropyleneimines, polyallylamines, chitosan, carboxyalkylcelluloses, aminoalkylcelluloses, maleic, fumaric and/or itaconic acid or anhydride polymers, polyamidoamines, and a mixture thereof.

The one or more film-forming polymers may be copolymers of (meth)acrylic acid and of at least one ester monomer of linear, branched or cyclic (meth)acrylic acid and/or of at least one amide monomer of linear, branched or cyclic, mono- or disubstituted (meth)acrylic acid; (meth)acrylic acid/tert-butyl(meth)acrylate and/or isobutyl (meth)acrylate/C₁-C₄ alkyl(meth)acrylate copolymers; (meth)acrylic acid/ethyl acrylate/methyl methacrylate terpolymers and tetrapolymers; methyl methacrylate/butyl or ethyl acrylate/hydroxyethyl or 2-hydroxypropyl acrylate or methacrylate/(meth)acrylic acid tetrapolymers; copolymers of acrylic acid and of C₁-C₄ alkyl methacrylate; terpolymers of vinylpyrrolidone, of acrylic acid and of C₁₋₂₀ alkyl methacrylate; amphoteric copolymers; vinyl esters of branched acids; vinyl esters of benzoic acid; copolymers of (meth)acrylic acid and of at least one olefinic monomer; copolymers of vinyl monoacid and/or of allylic monoacid; and a mixture thereof. In some cases, the one or more film-forming polymers include VP/dimethylaminoethylmethacrlate copolymer.

In some instances, the cleansing compositions do not require silicones, film-forming polymers, and sulfate-based surfactants. Thus, any one or more (or all) of these may optionally be excluded from the cleansing compositions. In other words, the compositions may be free or essentially free of silicones and/or film-forming polymers and/or sulfate-based surfactants. Nonetheless, in some instances, one or more silicones and/or one or more film-forming polymers and/or one or more sulfate-based surfactants may optionally be included in the cleansing compositions.

### Miscellaneous Thickening Agents

The cleansing compositions may contain one or more miscellaneous thickening agents other the cationic polymers and non-cationic polysaccharides of the present disclosure.

Non-limiting examples of miscellaneous thickening agents include olymeric thickening agents selected from ammonium polyacryloyldimethyl taurate, ammonium acryloyldimethyltaurate/VP copolymer, sodium polyacrylate, acrylates copolymers, polyacrylamide, carbomer, and acrylates/C10-30 alkyl acrylate crosspolymer.

In some instances, the miscellaneous thickening agent(s) are selected from carboxylic acid polymers (e.g., carbomer), crosslinked polyacrylate polymers, polyacrylamide polymers, and a mixture thereof.

In certain embodiments of the instant disclosure, the cleansing compositions include:
(a) about 4 to about 20 wt. %, preferably about 6 to about 15 wt. %, more preferably about 8 to about 13 wt. % of one or more anionic surfactants, salts thereof, and a mixture thereof, preferably selected from non-sulfate anionic surfactants; and more preferably selected from acyl isethionates, acyl sarcosinates, salts thereof, and a mixture thereof;
(b) about 0.5 to about 3 wt. %, preferably about 1 to about 2.5 wt. %, more preferably about 1.5 to about 2 wt. % of one or more fatty alcohols;
(c) about 0.1 to about 5 wt. %, preferably about 0.25 to about 4.5 wt. %, more preferably about 0.5 to about 4 wt. %, even more preferably about 0.75 to about 3.5 wt. % of one or more glyceryl esters selected from glyceryl stearate, glyceryl isostearate, and a mixture thereof;
(d) about 0.15 to about 1.2 wt. %, preferably about 0.2 to about 1 wt. %, more preferably about 0.25 to about 0.8 wt. %, even more preferably about 0.3 to about 0.7 wt. % of one or more cationic polymers, preferably selected from cationic guar gum derivatives, and more preferably selected from hydroxypropyl guar hydroxypropyltrimonium chloride, guar hydroxypropyltrimonium chloride, and a mixture thereof;
(e) about 0.5 to about 10 wt. %, preferably about 0.75 to about 8 wt. %, more preferably about 1 to about 6 wt. %, even more preferably about 1 to about 4 wt. % of one or more non-cationic polysaccharides, preferably selected from starches, gums, cellulose-based polymers, and a mixture thereof, and more preferably selected from hydroxypropyl starch phosphate, hydroxyethyl cellulose, xanthan gum, sclerotium gum, and a mixture thereof;
(f) about 50 to about 80 wt. % of water;
(g) optionally, one or more conditioning agents selected from non-silicone fatty compounds, silicones, hydrogenated starch hydrolysates, alkyl amines, and a mixture thereof, and preferably selected from hydrogenated starch hydrolysates in an amount of from about 1 to about 15 wt. %, preferably about 3 to about 14 wt. %, and more preferably about 5 to about 12 wt. %;
(h) optionally, one or more amphoteric surfactants selected from betaines, alkyl sultaines, alkyl amphoacetates, alkyl amphoproprionates, and a mixture thereof, and preferably selected from coco-betaines, cocamidopropyl betaine, and a mixture thereof, and present in an amount of from about 0.5 to about 10 wt. %, preferably about 1 to about 8 wt. %, and more preferably about 2 to about 6 wt. %; and
(i) optionally, one or more opacifying agent selected from clay, talc, bentonite, and a mixture thereof, and preferably present in an amount of from about 0.1 to about 8 wt. %, preferably about 0.25 to about 6 wt. %, and more preferably about 0.5 to about 4 wt. %;
wherein all weight percentages are based on the total weight of the cleansing composition.

In an embodiment of the instant disclosure, the cleansing compositions include:
(a) about 8 to about 12 wt. % of non-sulfate anionic surfactants, salts thereof, and a mixture thereof and selected from acyl isethionates, acyl sarcosinates, salts thereof, and a mixture thereof, and preferably selected from sodium cocoyl isethionate, sodium lauroyl methyl isethionate, sodium cocoyl methyl isethionate, sodium cocoyl sarcosinate, sodium lauroyl sarcosinate, and a mixture thereof;
(b) about 1.5 to about 2 wt. % of one or more fatty alcohols;
(c) about 0.75 to about 3.5 wt. % of one or more glyceryl esters selected from glyceryl stearate, glyceryl isostearate, and a mixture thereof;
(d) about 0.3 to about 0.7 wt. % of one or more cationic polymers selected from hydroxypropyl guar hydroxypropyltrimonium chloride, guar hydroxypropyltrimonium chloride, and a mixture thereof;
(e) about 1 to about 4 wt. % of one or more non-cationic polysaccharides selected from hydroxypropyl starch phosphate, hydroxyethyl cellulose, xanthan gum, sclerotium gum, and a mixture thereof;
(f) about 50 to about 80 wt. % of water;
(g) optionally, one or more conditioning agents selected from plant/vegetable oils and butters, silicones, starch hydrolysates, and a mixture thereof;
(h) optionally, one or more amphoteric surfactants; and
(i) optionally, one or more opacifying agent selected from clay, talc, bentonite, and a mixture thereof;
wherein all weight percentages are based on the total weight of the cleansing composition.

Additionally, as noted above, the cleansing compositions do not require silicones, film-forming polymers, and sulfate-based surfactants. Thus, any one or more (or all) of these may optionally be excluded from the cleansing compositions. In other words, the compositions may be free or essentially free of silicones and/or film-forming polymers and/or sulfate-based surfactants. Nonetheless, in some instances, one or more silicones and/or one or more film-forming polymers and/or one or more sulfate-based surfactants may optionally be included in the cleansing compositions.

When present, the sulfate-based surfactants may be selected from alkyl sulfates, alkyl ether sulfates, their salts, and mixtures thereof.

Suitable examples of sulfate-based surfactants are lauryl sulfates, lauryl ether sulfates, coco-sulfate, and/or their salts thereof (e.g., sodium, ammonium), and mixtures thereof.

The viscosity of the cleansing compositions discussed throughout the instant disclosure can vary but is often similar to that of typical cleansing, shampooing, and/or conditioning compositions. The viscosity measurements can also be carried out, for example, using a Brooksfield viscometer/rheometer using a RV-3 Disk spindle at a speed of 5, 10, 15, and/or 20 rpm or using a Rheomat with an M3 or an M4 spindle for a time period, such as at about 30 seconds. Accordingly, in some instances, the viscosity can be from about 10 uD to about 45 uD (or equivalent about 2400 cP to about 11,000 cp) at a temperature ranging from about 15° C. to about 35° C., such as at about 25° C., as measured with a Rheomat with an M4 spindle for a 30 seconds. Software may be employed for automated instrument control and data acquisition.

In some cases, the viscosity (M4 spindle) is from about 10 uD to about 45 uD (about 2400 cP to about 11,000 cP, centipoise), about 12 uD to about 44 uD, about 13 uD to about 42 uD, about 14 uD to about 40 uD, about 15 uD to about 38 uD, about 16 uD to about 36 uD, about 17 uD to about 35 uD, about 18 uD to about 34 uD, about 19 uD to about 32 uD, about 20 uD to about 30 uD, about 21 uD to about 28 uD, about 22 uD to about 27 uD, or about 23 uD to about 26 uD, including all ranges and subranges therebetween.

The texture and appearance of the cleansing compositions of the instant disclosure may also be assessed by a texture analyzer according to several parameters—hardness, adhesiveness and thickness. Such assessments are related to the cream-like texture and viscosity of the compositions of the instant disclosure. Thus, the hardness of the cleansing compositions of the instant disclosure may range from about 7 g to about 15 g, or about 7.5 g to about 12 g, or about 8 g to about 10 g, including all ranges and subranges therebetween, as measured by a texture analyzer; the thickness of the cleansing compositions of the instant disclosure may range from about 400 mj (millijoules) to about 900 mj, or about 420 mj to about 800 mj, or about 450 mj ml to about 700 ml mj, including all ranges and subranges therebetween, as measured by a texture analyzer; and the adhesiveness of the cleansing compositions of the instant disclosure may range from about 100 mj to about 300 mj, or about 110 mj to about 280 mj, or about 120 mj to about 250 mj, including all ranges and subranges therebetween, as measured by a texture analyzer.

The cleansing compositions described throughout the instant disclosure may be in a variety of different forms, for example, gels, lotions, creams, milks, sprays, and the like.

In an embodiment, the cleansing composition is in the form of an emulsion such as an oil in water emulsion.

In an embodiment, the cleansing composition is a cream in the form of an oil in water emulsion.

In an embodiment, the cleansing compositions may be in the form of a dispersion.

Due to the cleansing and conditioning properties of the cleansing compositions, in some instances, the cleansing compositions may be designated as a “shampoo,” a “conditioning shampoo,” an “all-in-one conditioning and shampooing composition,” a “cream shampoo,” a “cream rinse,” a “cleansing cream,” a “body cleanser (or body wash),” a “skin cleanser,”, a “facial cleanser,” “all-in-one skin or face conditioning and cleansing composition,” or a “cleansing scrub,” and variations thereof. The cleansing compositions may also be a body wash or both a hair and body wash.

The cleansing compositions of the instant disclosure are particularly useful for cleansing and conditioning keratinous substrates such as hair and skin.

Thus, in certain embodiments, the instant disclosure is directed to methods of treating keratinous substrates, the method comprising the steps of:
(1) contacting the keratinous substrates with a rinse-off cleansing composition comprising:
(a) about 4 to about 20 wt. %, preferably about 6 to about 15 wt. %, more preferably about 8 to about 12 wt. % of one or more anionic surfactants, salts thereof, and a mixture thereof, preferably selected from non-sulfate anionic surfactants; and more preferably selected from acyl isethionates, acyl sarcosinates, salts thereof, and a mixture thereof;
(b) about 0.5 to about 3 wt. %, preferably about 1 to about 2.5 wt. %, more preferably about 1.5 to about 2 wt. % of one or more fatty alcohols;
(c) about 0.1 to about 5 wt. %, preferably about 0.25 to about 4.5 wt. %, more preferably about 0.5 to about 4 wt. %, even more preferably about 0.75 to about 3.5 wt. % of one or more glyceryl esters selected from glyceryl stearate, glyceryl isostearate, and a mixture thereof;
(d) about 0.15 to about 1.2 wt. %, preferably about 0.2 to about 1 wt. %, more preferably about 0.25 to about 0.8 wt. %, even more preferably about 0.3 to about 0.7 wt. % of one or more cationic polymers, preferably selected from cationic guar gum derivatives, and more preferably selected from hydroxypropyl guar hydroxypropyltrimonium chloride, guar hydroxypropyltrimonium chloride, and a mixture thereof;
(e) about 0.5 to about 10 wt. %, preferably about 0.75 to about 8 wt. %, more preferably about 1 to about 6 wt. %, even more preferably about 1 to about 4 wt. % of one or more non-cationic polysaccharides, preferably selected from starches, gums, cellulose-based polymers, and a mixture thereof, and more preferably selected from hydroxypropyl starch phosphate, hydroxyethyl cellulose, xanthan gum, sclerotium gum, and a mixture thereof;
(f) about 50 to about 80 wt. % of water;
(g) optionally, one or more conditioning agents selected from non-silicone fatty compounds, silicones, hydrogenated starch hydrolysates, alkyl amines, and a mixture thereof, and preferably selected from hydrogenated starch hydrolysates in an amount of from about 1 to about 15 wt. %, preferably about 3 to about 14 wt. %, and more preferably about 5 to about 12 wt. %;
(h) optionally, one or more amphoteric surfactants selected from betaines, alkyl sultaines, alkyl amphoacetates, alkyl amphoproprionates, and a mixture thereof, and preferably selected from betaines, and more preferably selected from coco-betaines, cocamidopropyl betaine, and a mixture thereof, and present in an amount of from about 0.5 to about 10 wt. %, preferably about 1 to about 8 wt. %, and more preferably about 2 to about 6 wt. %; and
(i) optionally, one or more opacifying agent selected from clay, talc, bentonite, and a mixture thereof, and preferably present in an amount of from about 0.1 to about 8 wt. %, preferably about 0.25 to about 6 wt. %, and more preferably about 0.5 to about 4 wt. %;
wherein all weight percentages are based on the total weight of the cleansing composition; and
(2) rinsing the cleansing composition from the keratinous substrate.

Additionally, when used on hair, the cleansing compositions provide a variety of desirable cosmetic and styling benefits to the hair, for example, smoothness, detangling, and shine. Accordingly, the cleansing compositions are useful in methods for cleansing hair, methods of conditioning hair, and methods for imparting smoothness, detangling, and/or shine to hair. Accordingly, the instant disclosure encompasses methods for treating hair with the cleansing compositions of the instant disclosure. Such methods may include simply applying a cleansing composition of the instant disclosure to the hair.

In some cases, methods of using the cleansing compositions include shampooing and/or conditioning the hair with a cleansing composition of the instant disclosure. Such methods typically include applying an effective amount of a cleansing composition of the instant disclosure to the hair, allowing the cleansing composition to remain on the hair for a period of time, and subsequently rinsing the cleansing composition from the hair. The period of time for which the cleansing composition is allowed to remain on the hair is usually not long, e.g., not longer than about 5 minutes. Usually, the cleansing composition is merely allowed to remain on the hair for a period of time sufficient to incorporate the cleansing composition throughout the hair, for example, by lathering the composition throughout the hair using one's hands. The amount of time is sufficient for the cleansing composition to interact with the hair and any dirt, oil, contamination, etc., that may exist on the hair so that when rinsed, the dirt, oil, contamination, etc., can be effectively removed from the hair and the conditioning agents of the cleansing composition can interact with the hair to condition it. Thus, the cleansing composition may be allowed to remain on the hair for about 5 seconds to about 5 minutes, about 5 seconds to about 3 minutes, about 5 seconds to about 2 minutes, about 5 seconds to about 1 minute, about 30 seconds to about 5 minutes, or about 30 seconds to about 3 minutes.

As is common when using shampoo and/or conditioning compositions, the hair may be wetted or rinsed with water prior to application of a cleansing composition of the instant disclosure. Having water already in the hair can be helpful for creating lather when applying the cleansing compositions because the water interacts with the surfactants of the surfactant system.

Implementation of the present disclosure is provided by way of the following examples. The examples serve to illustrate the technology without being limiting in nature.

### EXAMPLES

### Example 1

TABLE 1
SHAMPOO FORMULATIONS

| | | Formula | | | | | | | | |
|---|---|---|---|---|---|---|---|---|---|---|
| | | EX 1 | EX 2 | EX 3 | EX 4 | EX 5 | EX 6 | EX 7 | EX 8 | EX 9 |
| Classifications | INCI US NAME | wt. % | wt. % | wt. % | wt. % | wt. % | wt. % | wt. % | wt. % | wt. % |
| Anionic | SODIUM COCOYL | 8.8 | 8.8 | 11.4 | 11.4 | 8.8 | 8.8 | 11.4 | 10 | 10 |
| surfactant(s) | ISETHIONATE | | | | | | | | | |
| Anionic | SODIUM COCO- | — | — | — | — | — | — | — | — | 3.2 |
| surfactant(s) | SULFATE | | | | | | | | | |
| Fatty | CETEARYL ALCOHOL | 1.5-2 | 1.5-2 | 1.5-2 | 1.5-2 | 1.5-2 | 1.5-2 | 1.5-2 | 1.5-2 | 1.5-2 |
| Alcohol(s) | | | | | | | | | | |
| Gylceryl | GLYCERYL STEARATE | 3 | 3 | 1 | 2 | 3 | 3 | 2− | 3 | 2− |
| Esters/ | AND/OR GLYCERYL | | | | | | | | | |
| Emulsifier(s) | ISOSTEARATE | | | | | | | | | |
| Cationic | HYDROXYPROPYL GUAR | 0.6 | 0.6 | 0.4 | 0.4 | 0.6 | 0.6 | 0.4 | 0.6 | 0.4 |
| Polymer(s) - | HYDROXYPROPYL- | | | | | | | | | |
| Cationic Guar | TRIMONIUM CHLORIDE | | | | | | | | | |
| Derivatives or | AND/OR GUAR | | | | | | | | | |
| Polyquaternium | HYDROXYPROPYL- | | | | | | | | | |
| compound(s) | TRIMONIUM CHLORIDE | | | | | | | | | |
| | POLYQUATERNIUM-37 | — | — | — | — | 0.5 | — | — | — | — |
| Non-cationic | HYDROXYPROPYL STARCH | 2.2 | — | 2.64 | 2.2 | 2.2 | — | 2.2 | 2.2 | 2.2 |
| polysaccharide(s) | PHOSPHATE AND/OR | | | | | | | | | |
| | HYDROXYETHYLCELLULOSE | | | | | | | | | |
| | AND/OR XANTHAN GUM | | | | | | | | | |
| Amphoteric | COCO-BETAINE (30 WT. %) | 10 | 10 | — | — | 10 | 10 | — | 15 | 10 |
| Surfactant(s) | AND SODIUM CHLORIDE | | | | | | | | | |
| | (6.5 WT. %) AND WATER | | | | | | | | | |
| Conditioning | BUTYROSPERMUM PARKII | 1 | 1 | 0.25 | 0.25 | 1 | 1 | 0.25 | 1 | 1 |
| Agent(s) - | (SHEA) BUTTER AND/OR | | | | | | | | | |
| Non-silicone | PERSEA GRATISSIMA | | | | | | | | | |
| oils (plant/ | (AVOCADO) OIL | | | | | | | | | |
| vegetable oils) | | | | | | | | | | |
| Conditioning | DIMETHICONE COPOLYMER | — | 0.9 | — | — | — | — | — | — | — |
| Agent(s) - | COMP[OUND | | | | | | | | | |
| Silicones | | | | | | | | | | |
| Conditioning | HYDROGENATED | 7 | — | 10.5 | 10.5 | 7 | — | 10.5 | 7 | 3.5 |
| Agent(s) - | STARCH HYDROLYSATE | | | | | | | | | |
| Starch | | | | | | | | | | |
| Hydrolystates | | | | | | | | | | |
| Opacifying | KAOLIN AND/OR TALC | — | — | 3 | 3 | — | — | 3 | — | 3 |
| Agent(s) | AND/OR BENTONITE | | | | | | | | | |
| Nonionic | PPG-1 TRIDECETH-6 | — | 0.06 | — | — | — | 0.07 | — | 0.07 | — |
| Surfactant(s) | AND/OR C12-13 | | | | | | | | | |
| | PARETH-3 AND/OR | | | | | | | | | |
| | C12-13 PARETH-23 | | | | | | | | | |
| Additive(s) | SDOIUM ISETHIONATE | ≤3 | ≤3 | ≤3 | ≤3 | ≤3 | ≤3 | ≤3 | ≤3 | ≤3 |
| | AND/OR PLANT EXTRACTS | | | | | | | | | |
| | AND/OR HYDROLYZED | | | | | | | | | |
| | PROTEINS AND/OR | | | | | | | | | |
| | FRAGRANCE(S) AND/OR | | | | | | | | | |
| | COLORANT(S) AND/OR | | | | | | | | | |
| | CHELATING AGENT(S) | | | | | | | | | |
| | AND/OR PRESERVATIVES | | | | | | | | | |
| | AND/OR PH ADJUSTORS | | | | | | | | | |
| | AND/OR VITAMINS, ETC. | | | | | | | | | |
| Water | GLYCERIN AND/OR | 2-5 | 2-5 | 2-5 | 2-5 | 2-5 | 2-5 | 2-5 | 2-5 | 2-5 |
| Soluble | CAPRYLYL GLYCOL | | | | | | | | | |
| Solvent(s) | | | | | | | | | | |
| Solvent | WATER | Q.S. | Q.S. | Q.S. | Q.S. | Q.S. | Q.S. | Q.S. | Q.S. | Q.S. |
| | | 100% | 100% | 100% | 100% | 100% | 100% | 100% | 100% | 100% |

TABLE 2
SHAMPOO FORMULATIONS

| | | Formula | | | | | | | |
|---|---|---|---|---|---|---|---|---|---|
| | | EX 10 | EX 11 | EX 12 | EX 13 | EX 14 | EX 15 | EX 16 | EX 17 |
| Classifications | INCI US NAME | wt. % | wt. % | wt. % | wt. % | wt. % | wt. % | wt. % | wt. % |
| Anionic | SODIUM COCOYL | 10 | 7 | 7 | 10 | 10 | 8.8 | 10 | 10 |
| surfactant(s) | ISETHIONATE | | | | | | | | |
| | SODIUM LAUROYL | 2.7 | 2.7 | 2.7 | 2.7 | 2.7 | 2.7 | 2.7 | 2.7 |
| | SARCOSINATE | | | | | | | | |
| Fatty Alcohol(s) | CETEARYL ALCOHOL | 1.5-2 | 1.5-2 | 1.5-2 | 1.5-2 | 1.5-2 | 1.5-2 | 1.5-2 | 1.5-2 |
| Glyceryl | GLYCERYL STEARATE | 3 | 3 | 3 | 3 | 3 | 3 | 3 | 3 |
| Esters/ | AND/OR GLYCERYL | | | | | | | | |
| Emulsifier(s) | ISOSTEARATE | | | | | | | | |
| Cationic | HYDROXYPROPYL GUAR | 0.6 | 0.6 | 0.6 | 0.6 | 0.6 | 0.6 | 0.6 | 0.6 |
| Polymer(s) - | HYDROXYPROPYL- | | | | | | | | |
| Cationic Guar | TRIMONIUM CHLORIDE | | | | | | | | |
| Derivatives or | AND/OR GUAR | | | | | | | | |
| Polyquaternium | HYDROXYPROPYL- | | | | | | | | |
| compound(s) | TRIMONIUM CHLORIDE | | | | | | | | |
| Non-cationic | HYDROXYPROPYL STARCH | 1.1 | 1.1 | 1.1 | 1.1 | 1.1 | 1.1 | 1.1 | 1.1 |
| polysaccharide(s) | PHOSPHATE AND/OR | | | | | | | | |
| | HYDROXYETHYLCELLULOSE | | | | | | | | |
| | AND/OR XANTHAN GUM | | | | | | | | |
| Amphoteric | COCO-BETAINE (30 WT. %) | 9 | 9 | 9 | 9 | 9 | 9 | 9 | 9 |
| Surfactant(s) | AND SODIUM CHLORIDE | | | | | | | | |
| | (6.5 WT. %) AND WATER | | | | | | | | |
| Conditioning | BUTYROSPERMUM PARKII | 1 | 0.5 | 0.5 | 0.5 | 1 | 1 | 1 | 1 |
| Agent(s) - Non- | (SHEA) BUTTER AND/OR | | | | | | | | |
| silicone oils | PERSEA GRATISSIMA | | | | | | | | |
| (plant/vegetable | (AVOCADO) OIL | | | | | | | | |
| oils) | | | | | | | | | |
| Conditioning | HYDROGENATED | 7 | 7 | 7 | 7 | 7 | 7 | 7 | 7 |
| Agent(s) - | STARCH HYDROLYSATE | | | | | | | | |
| Starch | | | | | | | | | |
| Hydrolystates | | | | | | | | | |
| Opacifying | KAOLIN AND/OR TALC | — | — | — | — | — | — | 0.1 | — |
| Agent(s) | AND/OR BENTONITE | | | | | | | | |
| Additive(s) | SDOIUM ISETHIONATE | ≤3 | ≤3 | ≤3 | ≤3 | ≤3 | ≤3 | ≤3 | ≤3 |
| | AND/OR PLANT EXTRACTS | | | | | | | | |
| | AND/OR HYDROLYZED | | | | | | | | |
| | PROTEINS AND/OR | | | | | | | | |
| | FRAGRANCE(S) AND/OR | | | | | | | | |
| | COLORANT(S) AND/OR | | | | | | | | |
| | CHELATING AGENT(S) | | | | | | | | |
| | AND/OR PRESERVATIVES | | | | | | | | |
| | AND/OR PH ADJUSTORS | | | | | | | | |
| | AND/OR VITAMINS, ETC. | | | | | | | | |
| Water Soluble | GLYCERIN AND/OR | 2-5 | 2-5 | 2-5 | 2-5 | 2-5 | 2-5 | 2-5 | 2-5 |
| Solvent(s) | CAPRYLYL GLYCOL | | | | | | | | |
| Solvent | WATER | Q.S. | Q.S. | Q.S. | Q.S. | Q.S. | Q.S. | Q.S. | Q.S. |
| | | 100% | 100% | 100% | 100% | 100% | 100% | 100% | 100% |

The formulas of the invention were prepared according to the general process steps as follows:
1. Hydrate starch or other powder-like thickeners such as the non-cationic polysaccharides (nonionic gum, hydroxyethylcellulose) with water in a side phase.
2. In a main phase, add the remainder of water and the cationic thickening agent, as well as any desired hydrogenated starch hydrolysates or organic solvents.
3. Begin heating the main phase to 70 C. At given temperature, add fatty alcohol, surfactants, and other water soluble ingredients, conditioning agents, opacifying agents.
4. Begin cooling to room temperature.
5. At a given temperature, add preservatives.
6. Add the side phase and homogenize.
7. Sweep until the mixture is at room temperature.

The inventive formulas were found to be high foaming and cream-like and with a high viscosity. In addition, the thick consistency of the inventive formulas allowed for the suspension of particles from clays, powders, colorants and scrubs.

The formulas were observed to have a matte texture (or non-pearlescent) when a betaine surfactant which was considered to indicate “natural” or “naturality” and conditioning. The consistency of the formulas was thick yet easy to break and melt upon application so that the formulas were easily applied onto hair. When applied on hair, the inventive formulas produced a mass effect which is an indication of the thickness of the hair, that is, an effect that provides a feeling of a greater mass of hair or thicker hair. The formulas also provided conditioning effects on hair.

The shampoo formulas of the invention (inventive formulas) were thick enough so that they did not easily drip and can be packaged in a jar which has the advantages of better portablility and product use.

### Example 2 Physical Properties of Formulations

The physical properties of the formulas of the invention were evaluated with respect to viscosity and texture and compared to those of conventional hair masque (mask) or hair treatment products that are generally thicker or more viscous than conventional hair rinse-off shampoos and conditioners. Hair mask compositions are also typically emulsion types of formulations.

Viscosity was measured on a Rheomat using a selected spindle size (M2 to M4). A higher spindle size for measuring is required for more viscous formulations. Texture was measured using a Brookfield CT3 Texture Analyzer with a TA43 probe. The texture was evaluated according to: (i) hardness, which refers to how firm the texture is or is related to the amount of force needed to attain a given deformation of the test surface; (ii) thickness, which refers to the consistency of the formula during and after penetration of probe; and (iii) adhesiveness, which indicates how tacky the texture is.

TABLE 3
CONVENTIONAL HAIR MASK FORMULAS

| | | Conventional | Conventional |
|---|---|---|---|
| | | Thick Rinse-out | Light Rinse-out |
| Classifications | INCI US NAME | Hair Mask EX C1 | Hair Mask EX C2 |
| Cationic surfactant(s) | BEHENTRIMONIUM CHLORIDE | 23.04 | 4.1 |
| | AND/OR CETRIMONIUM | | |
| | CHLORIDE AND/OR | | |
| | QUATERNIUM-87 | | |
| Fatty Alcohol(s) | STEARYL ALCOHOL AND/OR | 7 | 2 |
| | CETEARYL ALCOHOL | | |
| Other fatty compounds(s) - | CAPRYLIC/CAPRIC | 3.5 | 1.4 |
| esters, triglycerides, | TRIGLYCERIDE AND/OR CETYL | | |
| ceramides | ESTERS AND/OR PALNT WAX | | |
| | AND/OR CERAMIDE | | |
| Non-cationic | HYDROXYPROPYL GUAR | 0.15 | 4.4 |
| polysaccharide(s) | AND/OR (HYDROXYPROPYL | | |
| | STARCH PHOSPHATE | | |
| Nonionic surfactant(s) | TRIDECETH-9 | | 0.004 |
| Silicone | AMODIMETHICONE | 2.3 | 1.7 |
| Additive(s) | PLANT EXTRACTS AND/OR | ≤3 (1.9) | ≤3 (2.1) |
| | HYDROLYZED PROTEINS | | |
| | AND/OR FRAGRANCE(S) | | |
| | AND/OR COLORANT(S) AND/OR | | |
| | CHELATING AGENT(S) AND/OR | | |
| | PRESERVATIVES AND/OR PH | | |
| | ADJUSTORS AND/OR VITAMINS, | | |
| | ETC. | | |
| Water Soluble Solvent(s) | GLYCERIN AND/OR CAPRYLYL | 1.7 | 1.3 |
| | GLYCOL AND/OR PROPYLENE | | |
| | GLYCOL | | |
| Solvent | WATER | Q.S. 100% | Q.S. 100% |
| | | (80.4) | (83) |

TABLE 4
PHYSICAL PROPERTY

| | EX 1 | EX 2 | EX 4 | EX C1 | EX C2 |
|---|---|---|---|---|---|
| VISCOSITY* (RHEOMAT, | 23.5 | 25.3 | 24.6 | 27 | 20 |
| M4, uD, Units of Deflection) | | | | | |
| TEXTURE ANALYZER** | 9 | 9 | 8.5 | 14.5 | 8.5 |
| (HARDNESS IN g) | | | | | |
| TEXTURE ANALYZER** | 609.8 | 699.3 | 465 | 895.5 | 698 |
| (THICKNESS, MJ, | | | | | |
| MILLIJOULES) | | | | | |
| TEXTURE ANALYZER** | 141.4 | 175.3 | 184.8 | 280.5 | 150 |
| (ADHESIVENESS, MJ) | | | | | |

*viscosity was measured with spindle 4 (M4) on rheomat. The higher the value as measured in uD, the higher the viscosity
**Brookfield CT3 Texture analyzer with TA43 probe and 30 ml of product

Conclusion: Due to the thick textures of the test formulas, a spindle size of M4 was used to measure the viscosities of both the invention formulas and the conventional hair masks. It was found that the viscosities of the inventive formulas EX 1, EX 2, and EX 4 were at least about 23 uD which is in the same range as traditional/conventional rinse-off hair masks, EX C1 and EX C2. Both inventive and conventional hair mask formulas were thicker and creamier textures (20 to 27 uD) as compared to traditional or conventional shampoo compositions which are typically aqueous, non-creamy or non-emulsion types. For a traditional sulfate-based shampoo, the typical viscosity as measured by the Rheomat is 49.7 uD using spindle M3 because the viscosity was too low to be measured with spindle M4.

The hardness, thickness and adhesiveness of the inventive formulas were also found to be comparable to or within the range of those of the conventional hair mask formulas.

### Example 3 Testing Shampoo with Conditioner (Bundle Test)

Typically, shampoos are part of a cleansing or cleansing/conditioning routine or system for use on hair. Such routines or systems include a rinse-off shampoo, a rinse-off conditioner, and optionally, other rinse-off or leave-in hair treatment or hair masque products for additional conditioning, treatment or other desired performance benefits. In particular, shampoos are employed on hair as a first step in such routines, followed by rinsing the hair with water, then by a second step of treating the hair with a conditioner. This shampoo-conditioner system may also be referred to as a bundle.

### Bundle Test A

TABLE 5
CONDITIONER FORMULATION (CONDITIONER 1) TESTED
WITH THE INVENTIVE SHAMPOO IN A BUNDLE TEST

| | | CONDITIONER |
|---|---|---|
| Classifications | INCI US | 1 Wt. % |
| Cationic Surfactant(s) | BEHENTRIMONIUM CHLORIDE and/or | 2-3 |
| | CETROMONIUM CHLORIDE | |
| Fatty Alcohol(s) | CETEARYL ALCOHOL | 5-8 |
| Fatty Substance(s)/ | PLANT/VEGETABLE OILS | 1-5 |
| conditioning agents | | |
| Additive(s) | FRAGRANCE(S), COLORANT(S), CHELATING | ≤3 |
| | AGENT(S), PRESERVATIVES, AND/OR PH | |
| | ADJUSTORS, UV FILTERS, ETC. | |
| Water Soluble Solvent(s) | GLYCERIN, ISOPROPYL ALCOHOL | 0.5-2 |
| Solvent | WATER | Q.S. 100% |

### Bundle Test B

TABLE 6
COMPARATIVE SHAMPOO FORMULATION USED WITH A CONDITIONER
FORMULATION (CONDITIONER 2) IN A BUNDLE TEST

| | | COMPARATIVE |
|---|---|---|
| | | SHAMPOO |
| Classifications | INCI US | wt. % |
| Anionic surfactant(s) | SODIUM COCOYL ISETHIONATE, | 20 |
| | SODIUM LAUROYL SARCOSINATE, | |
| | SODIUM LAURYL SULFOACETATE | |
| Fatty Alcohol(s) | CETEARYL ALCOHOL | — |
| Emulsifier(s) | GLYCERYL STEARATE, | — |
| | GLYCERYL ISOSTEARATE | |
| Cationic Polymer(s) - | HYDROXYPROPYL GUAR | — |
| Cationic Guar Derivatives | HYDROXYPROPYLTRIMONIUM | |
| | CHLORIDE, GUAR | |
| | HYDROXYPROPYLTRIMONIUM | |
| | CHLORIDE | |
| Cationic Polymer(s) | POLYQUATERNIUM-7, | 0.7 |
| | POLYQUATERNIUM-10, | |
| | HEXADIMETHRINE CHLORIDE | |
| Thickening Agent(s) - | CARBOMER, ACRYLATES | 0.6 |
| Acrylic/acrylate types | COPOLYMER | |
| Amphoteric Surfactant(s) | COCO-BETAINE | 2.1 |
| Conditioning Agent(s) - | CERAMIDE, PEG-55 PROPYLENE | 0.2 |
| Non-silicone oils, | GLYCOL OLEATE | |
| esters | | |
| Conditioning Agent(s) - | DIVINYLDIMETHICONE/DIMETHICONE | 0.85 |
| Silicones | COPOLYMER, AMODIMETHICONE | |
| Conditioning Agent(s) - | HYDROGENATED STARCH | — |
| Starch Hydrolystates | HYDROLYSATE | |
| Opacifying Agent(s) | GLYCOL DISTEARATE | 1.8 |
| Nonionic Surfactant(s) | PPG-5-CETETH-20, GLYCERETH-26, | 2.7 |
| | DECYL GLUCOSIDE, C12-13 PARETH-3, | |
| | C12-13 PARETH-23, C11-15 PARETH-7, | |
| | LAURETH-9, TRIDECETH-12 | |
| Additive(s) | SALT(S) (SODIUM CHLORIDE, SODIUM | ≤5 |
| | ISETHIONATE), FRAGRANCE(S), | |
| | COLORANT(S), CHELATING AGENT(S), | |
| | PRESERVATIVES, AND/OR PH | |
| | ADJUSTORS, PLANT EXTRACTS, | |
| | MALTODEXTRIN, ETC. | |
| Water Soluble Solvent(s) | GLYCERIN, PROPYLENE GLYCOL | 0.27 |
| Solvent | WATER | Q.S. 100% |

TABLE 7
CONDITIONER FORMULATION USED WITH THE
COMPARATIVE SHAMPOO IN A BUNDLE TEST

| | | CONDITIONER |
|---|---|---|
| Classifications | INCI US | 2 Wt. % |
| Cationic Surfactant(s) | BEHENTRIMONIUM CHLORIDE and/or | 2-3 |
| | CETROMONIUM CHLORIDE | |
| Cationic Polymer(s) | HEXADIMETHRINE CHLORIDE | 0.01-0.1 |
| Fatty Alcohol(s) | CETEARYL ALCOHOL | 5-8 |
| Fatty Substance(s) | PLANT/VEGETABLE OILS | 1-5 |
| Silicone(s) | AMODIMETHICONE | 0.5-2 |
| Additive(s) | FRAGRANCE(S), COLORANT(S), CHELATING | ≤3 |
| | AGENT(S), PRESERVATIVES, AND/OR PH | |
| | ADJUSTORS, UV FILTERS, MALTODEXTRIN, | |
| | ETC. | |
| Water Soluble Solvent(s) | ISOPROPYL ALCOHOL | 0.5-2 |
| Solvent | WATER | Q.S. 100% |

Conclusion:

The hair treated with the inventive bundle was found to have properties of greater ease of combing dry hair, a smooth appearance and a wet coating feel. The foregoing description illustrates and describes the invention. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−5% of the indicated number.

Some of the various categories of components identified for the hair-treatment compositions may overlap. In such cases where overlap may exist and the composition/product includes two overlapping components (or more than two overlapping components), an overlapping component does not represent more than one component. For example, a fatty acid may be defined as both a “fatty compound” and a “surfactant/emulsifier.” If a particular composition/product includes both a fatty compound component and an emulsifier component, a single fatty acid can serve as only a fatty compound or a surfactant/emulsifier (a single fatty acid does not serve as both the fatty compound and the surfactant/emulsifier).

All percentages, parts and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

The term “surfactants” includes salts of the surfactants even if not explicitly stated. In other words, whenever the disclosure refers to a surfactant, it is intended that salts of the surfactant are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants. However, additional cations such as ammonium ions, or alkanolammonium ions such as monoethanolammonium or triethanolammonium ions, may also form salts of surfactants.

The term “substantially free” or “essentially free” as used herein means the specific material may be present in small amounts that do not materially affect the basic and novel characteristics of the claimed invention. For instance, there may be less than 1% by weight of a specific material added to a composition, based on the total weight of the compositions (provided that an amount of less than 1% by weight does not materially affect the basic and novel characteristics of the claimed invention. Similarly, the compositions may include less than 0.5 wt. %, less than 0.1 wt. %, less than 0.05 wt. %, or less than 0.01 wt. %, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A rinse-off cleansing composition comprising:
(a) one or more anionic surfactants, salts thereof, and a mixture thereof;
(b) about 0.5 to about 3 wt. % of one or more fatty alcohols;
(c) about 0.1 to about 5 wt. %, of one or more glyceryl esters;
(d) about 0.15 to about 1.2 wt. % of one or more cationic polymers selected from cationic cellulose derivatives, quaternized hydroxyethyl cellulose, cationic starch derivatives, cationic guar gum derivatives copolymers of acrylamide and dimethyldiallyammonium chloride, polyquaterniums, and a mixture thereof;
(e) about 0.5 to about 10 wt. % of one or more non-cationic polysaccharides selected from starches, gums, cellulose-based polymers, and a mixture thereof; and
(f) water;
wherein all weight percentages are based on the total weight of the cleansing composition.

2. The cleansing composition of claim 1, comprising one or more anionic surfactants selected from non-sulfate anionic surfactants.

3. The cleansing composition of claim 2, comprising non-sulfate anionic surfactants selected from acyl isethionate(s), acyl sarcosinate(s), salts thereof, and a mixture thereof.

4. The cleansing composition of claim 1, wherein the anionic surfactants are present in an amount of from about 4 to about 20 wt. %, based on the total weight of the cleansing composition.

5. The cleansing composition of claim 1, comprising one or more fatty alcohols selected from cetyl alcohol, stearyl alcohol, cetearyl alcohol (mixture of cetyl alcohol and stearyl alcohol), octyldodecanol, 2-butyloctanol, 2-hexyldecanol, 2-undecylpentadecanol, oleic alcohol, linoleic alcohol, behenyl alcohol, 2-dodecylhexadecanol, 2-tetradecyl-1-octadecanol, 2-tetradecyl-1-eicosanol, 2-hexadecyl-1-octadecanol and 2-hexadecyl-1-eicosanol, octyldodecanol, 2-butyloctanol, 2-hexyldecanol, 2-undecylpentadecanol, oleyl alcohol or linoleyl alcohol, isostearyl alcohol, and a mixture thereof.

6. The cleansing composition of claim 1, comprising one or more glyceryl esters selected from glyceryl stearate, glyceryl isostearate, glyceryl stearate, glyceryl isostearate, glyceryl oleate, glyceryl caprate, and glyceryl caprylate and a mixture thereof.

7. The cleansing composition of claim 1, comprising one or more cationic polymers selected from one or more cationic guar gum derivatives.

8. The cleansing composition of claim 1, further comprising: (g) one or more conditioning agents selected from non-silicone fatty compounds, silicones, starch hydrolysates, alkyl amines, cationic polymers other than (d), cationic proteins and cationic protein hydrolysates and a mixture thereof.

9. The cleansing composition of claim 8, wherein the one or more conditioning agents include hydrogenated starch hydrolysates present in an amount of from about 1 to about 15 wt. %, based on the total weight of the cleansince composition.

10. The cleansing composition of claim 1, further comprising: (h) one or more amphoteric surfactants selected from betaines, alkyl sultaines, alkyl amphoacetates, alkyl amphoproprionates, and a mixture thereof.

11. The cleansing composition of claim 1, further comprising: (i) one or more opacifying agents selected from clay, kaolin, talc, bentonite, and a mixture thereof.

12. The cleansing composition of claim 1, wherein the composition is essentially free of sulfate-based surfactants, salts thereof, and a mixture thereof.

13. The cleansing composition of claim 1, further comprising one or more sulfate anionic surfactants selected from alkyl sulfates, alkyl ether sulfate, salts thereof, and a mixture thereof.

14. The cleansing composition of claim 1 having a viscosity of from about 10 uD to about 45 uD.

15. The cleansing composition of claim 1 having a hardness of from about 7 g to about 15 g, or about 7.5 g to about 12 g, or about 8 g to about 10 g, as measured by a texture analyzer.

16. The cleansing composition of claim 1 having a thickness of from about 400 mj (millijoules) to about 900 mj, or about 420 mj to about 800 mj, or about 450 mj to about 700 mj, as measured by a texture analyzer.

17. The cleansing composition of claim 1 having an adhesiveness of from about 100 ml to about 300 ml, or about 110 ml to about 280 ml, or about 120 ml to about 250 ml, as measured by a texture analyzer.

18. The cleansing composition of claim 1, wherein the composition is a shampoo composition for cleansing hair.

19. The cleansing composition of claim 1, wherein the composition is an oil in water emulsion.

20. A cleansing composition comprising:
(a) about 6 to about 15 wt. % of non-sulfate anionic surfactants, salts thereof, and a mixture thereof and selected from sodium cocoyl isethionate, sodium lauroyl methyl isethionate, sodium cocoyl methyl isethionate, sodium cocoyl sarcosinate, sodium lauroyl sarcosinate, and a mixture thereof;
(b) about 1 to about 2.5 wt. % of one or more fatty alcohols;
(c) about 0.25 to about 4.5 wt. % of one or more glyceryl esters selected from glyceryl stearate, glyceryl isostearate, and a mixture thereof;
(d) about 0.2 to about 1 wt. % of one or more cationic polymers selected from hydroxypropyl guar hydroxypropyltrimonium chloride, guar hydroxypropyltrimonium chloride, polyquaterniums, and a mixture thereof;
(e) about 0.75 to about 8 wt. % of one or more non-cationic polysaccharides selected from hydroxypropyl starch phosphate, hydroxyethyl cellulose, xanthan gum, sclerotium gum, and a mixture thereof;
(f) about 50 to about 80 wt. % of water;
(g) optionally, one or more conditioning agents selected from plant/vegetable oils and butters, silicones, starch hydrolysates, and a mixture thereof;
(h) optionally, one or more amphoteric surfactants; and
(i) optionally, one or more opacifying agent selected from clay, talc, bentonite, and a mixture thereof;
wherein all weight percentages are based on the total weight of the cleansing composition.

21. A method for treating keratinous substrates, the method comprising the steps of:
(1) contacting the keratinous substrates with a rinse-off cleansing composition comprising:
(a) one or more anionic surfactants, salts thereof, and a mixture thereof;
(b) about 0.5 to about 3 wt. % of one or more fatty alcohols;
(c) about 0.1 to about 5 wt. %, of one or more glyceryl esters;
(d) about 0.15 to about 1.2 wt. % of one or more cationic polymers selected from cationic cellulose derivatives, quaternized hydroxyethyl cellulose, cationic starch derivatives, cationic guar gum derivatives copolymers of acrylamide and dimethyldiallyammonium chloride, polyquaterniums, and a mixture thereof;
(e) about 0.5 to about 10 wt. % of one or more non-cationic polysaccharides, selected from starches, gums, cellulose-based polymers, and a mixture thereof; and
(f) water;
wherein all weight percentages are based on the total weight of the cleansing composition; and
(2) rinsing the cleansing composition from the keratinous substrate.

22. The method of claim 21, wherein the cleansing composition comprises one or more anionic surfactants selected from non-sulfate anionic surfactants.

23. The method of claim 22, wherein the cleansing composition comprises non-sulfate anionic surfactants selected from acyl isethionates, acyl sarcosinates, salts thereof, and a mixture thereof.

24. The method of claim 21, wherein the cleansing composition comprises anionic surfactants present in an amount of from about 4 to about 20 wt. %, based on the total weight of the cleansing composition.

25. The method of claim 21, wherein the cleansing composition comprises one or more glyceryl esters selected from glyceryl stearate, glyceryl isostearate, glyceryl stearate, glyceryl isostearate, glyceryl oleate, glyceryl caprate, and glyceryl caprylate and a mixture thereof.

26. The method of claim 21, wherein the cleansing composition comprises one or more cationic polymers selected from one or more cationic guar gum derivatives.

27. The method of claim 21, wherein the cleansing composition comprises further comprises: (g) one or more conditioning agents selected from non-silicone fatty compounds, silicones, starch hydrolysates, alkyl amines, cationic polymers other than (d), cationic proteins and cationic protein hydrolysates and a mixture thereof.

28. The method of claim 21, wherein the one or more conditioning agents include hydrogenated starch hydrolysates.

29. The method of claim 21, wherein the cleansing composition further comprises: (h) one or more amphoteric surfactants selected from betaines, alkyl sultaines, alkyl amphoacetates, alkyl amphoproprionates, and a mixture thereof.

30. The method of claim 21, wherein the cleansing composition further comprises: (i) one or more opacifying agents selected from clay, kaolin, talc, bentonite, and a mixture thereof.

31. The method of claim 21, wherein the cleansing composition is essentially free of sulfate-based surfactants, salts thereof, and a mixture thereof.

32. The method of claim 21, wherein the cleansing composition has a viscosity of from about 10 uD to about 11,000 uD.

33. The method of claim 21, wherein the cleansing composition has one or more of the following: a hardness of from about 8 g to about 10 g as measured by a texture analyzer; a thickness of about 400 mj to about 850 mj as measured by a texture analyzer; an adhesiveness of about 100 mj to about 250 mj as measured by a texture analyzer; and combinations thereof.

34. The method of claim 21, wherein the cleansing composition is a shampoo composition and the keratinous substrate is hair.
